(19) United States
(12) Patent Application Publication
(10) Pub. No.: US 2016/0228056 A1
(43) Pub. Date: Aug. 11, 2016

(54) DYNAMIC POSITRON EMISSION TOMOGRAPHY IMAGING

(71) Applicant: THE GENERAL HOSPITAL CORPORATION, Boston, MA (US)

(72) Inventors: Jacob M. Hooker, Charlestown, MA (US); Marjorie Villien, Charlestown, MA (US)

(21) Appl. No.: 15/022,373

(22) PCT Filed: Sep. 17, 2014
(86) PCT No.: PCT/US14/56068

§ 371 Date: Mar. 16, 2016

Related U.S. Application Data
(60) Provisional application No. 61/878,942, filed on Sep. 17, 2013.

(51) Int. Cl. A61B 5/00; G01R 33/48; A61B 5/055; A61B 6/03; A61B 6/00

(52) U.S. Cl. CPC A61B 5/4866; A61B 6/037; A61B 6/501; A61B 5/0042; A61B 5/055; G01R 33/481

(57) ABSTRACT

An emission tomography system and method are used to acquire a series of medical images of a subject during an imaging process using a radiotracer. The system includes detectors for acquiring gamma rays emitted from the subject after administration of the radiotracer. A stimulus system tracks a stimulus associated with the subject during imaging. A data processing system receives signals from the detectors and stimulus feedback from the stimulus system and correlates the signals with information about the stimulus associated with the subject. A reconstruction system receives the correlated signals and information about the stimulus from the data processing system and reconstructs a series of medical images of the subject showing a physiological response of the subject to the stimulus over a time period.

### CROSS-REFERENCES TO RELATED APPLICATIONS

This application claims priority to U.S. Patent Application No. 61/878,942 filed Sep. 17, 2013.

### STATEMENT REGARDING FEDERALLY SPONSORED RESEARCH

This invention was made with government support under grant numbers P41EB015896, S10RR019933, and R01EB014894 awarded by National Institutes of Health. The U.S. government has certain rights in the invention.

### BACKGROUND OF THE INVENTION

1. Field of the Invention

The invention relates to a positron emission tomography system and method that are used to acquire a series of medical images of a subject during an imaging process. The series of medical images of the subject show a physiological response of the subject to a behavioral paradigm stimulus over a time period.

2. Description of the Related Art

Although the brain represents only 2% of the body weight, it receives 15% of the cardiac output, 20% of total body oxygen consumption, and 25% of total body glucose utilization. Glucose utilization in the human brain, both at rest and during cognitive tasks, has been studied over the past 40 years using 2-[¹⁸F]-fluoro-2-deoxyglucose (FDG) with positron emission tomography (PET). This method, now widely used clinically, can provide a quantitative measurement of glucose uptake/utilization (herein, we will refer to this multifaceted measurement as glucose utilization). The availability of PET clinically has been largely dictated by the distribution network of ¹⁸FDG estimated in 2005 to capture more than 97% of the United States population.

The availability of FDG-PET expanded dramatically when it was demonstrated as a critical tool for diagnosis and staging in oncology. While the growth of PET and the majority of its current use can be attributed to oncology, historically PET was initially used as the first non invasive measurement of cerebral glucose utilization. In addition to the clinical utility of FDG-PET for applications such as epilepsy and Alzheimer's disease, the primary use of FDG in the brain has been in neuroscience research. However, temporal information has been essentially ignored in all human FDG studies.

There are hundreds of published neuroscience research studies that used FDG as surrogate marker of brain glucose utilization. These studies can be broken into cross-sectional and longitudinal designs. A “double bolus” method has been proposed to measure cerebral glucose utilization in two different physiological states in the same session (see, Nishizawa et al., “Double-injection FDG method to measure cerebral glucose metabolism twice in a single procedure”, Ann. Nucl. Med. 2001 June; 15(3):203-7). However, all 2-scan-protocols (i.e., 2 injections) are limited by the simple fact that data represent the integral of FDG uptake and utilization over 20-40 minutes following a bolus injection. These bolus methods fundamentally limit the temporal information of studies. For instance, the bolus method does not allow paradigms that compare the regional uptake and magnitude of FDG within subjects and within sessions using multiple tasks that might be expected to subtly influence relative glucose utilization.

FDG uptake and utilization is commonly simplified into a model where: (1) FDG is transported like glucose across membranes; (2) the transport is typically not considered rate limiting; and (3) phosphorylation from FDG by hexokinase is irreversible and traps radioactivity at the site of utilization by preventing further biochemical modification and export (on the time scale of imaging). A metabolic rate (CMR_{glu}) can be estimated mathematically using this model and with a number of imaging designs including “autoradiographic” static approaches and dynamic methods (e.g., Patlak models). The dynamic method, however, is a misnomer in that dynamic information about CMR_{glu} is not measured. Dynamic only refers to the acquisition of dynamic imaging data (short frames) and for analysis of one “averaged” value over the course of a scan.

Because no method exists to measure dynamic changes in glucose utilization, functional imaging with high temporal resolution is almost exclusively accomplished using functional magnetic resonance imaging of the BOLD signal (blood oxygen level dependent). Functional magnetic resonance imaging (fMRI) has excellent temporal resolution and can provide relative measurements of BOLD signal which arises from changes in the ratio of oxy- and deoxyhemoglobin which directly affect T₂*contrast. Changes in this ratio in response to neural activity arise from the interplay of changes in cerebral blood flow (CBF), cerebral blood volume (CBV), and cerebral metabolic rate of oxygen consumption (CMRO₂) mediated by neurovascular and neurometabolic coupling. This complex mechanism is not fully understood and highly multiparametric. However, the BOLD signal can be used to measure brain activity with 1-3 second time resolution.

Functional magnetic resonance imaging has excellent temporal resolution and can provide relative measurements of BOLD signal changes and/or cerebral blood flow using arterial spin labeling (ASL) techniques. Unfortunately, fMRI measurements using BOLD signal, which is the most robust and reliable fMRI contrast, are not quantitative in an absolute sense. ASL can provide a quantitative index (CBF) but has a poor signal to noise ratio. Thus, while fMRI is often a method of choice for brain mapping, there are disadvantages such as fMRI's reliance on indirect hemodynamic measures of neuronal activity and the lack of direct quantitative information.

Therefore, there exists a need for an alternative functional medical imaging system and method with high temporal resolution.

### SUMMARY OF THE INVENTION

The invention meets the foregoing needs by providing a positron emission tomography system and method that are used to acquire a series of medical images of a subject during an imaging process. The system includes detectors for acquiring gamma rays emitted from the subject after administration of a radiotracer such as FDG. A stimulus system tracks a stimulus associated with the subject during imaging. A data processing system receives signals from the detectors and stimulus feedback from the stimulus system and correlates the signals with information about the stimulus associated with the subject. A reconstruction system receives the correlated signals and information about the stimulus from the data processing system and reconstructs a series of medical images of the subject showing a physiological response of the subject to a behavioral paradigm stimulus over a time period.

It is an advantage of the invention to capture quantitative glucose utilization information while providing temporal glucose utilization dynamics. We have developed a novel FDG imaging method for use with PET. By infusing FDG at a constant rate, we access the temporal information that is not available through a bolus method. This new technique provides true dynamic FDG imaging with temporal resolution in the order of the minute. The ‘dynamic’ method that already exists is a misnomer in that dynamic information about CMR_{glu} is not measured. Dynamic only refers to the acquisition of dynamic imaging data (short frames) and for analysis of one “averaged” value over the course of a scan. The method disclosed herein is truly dynamic in that changes in glucose utilization can be determined at multiple times within a scan session. Our new method is a truly dynamic tool for the measure of glucose use in the human brain with imaging.

It is another advantage of the invention to provide a time-resolved method for glucose use monitoring in the human brain. The method is versatile in design and thus can be used for many applications. For example, the methods described herein can be used with existing PET scanning infrastructure.

The methods described herein: (i) allow characterization of functional changing in glucose utilization during brain activity using multi-task within-subject designs; (ii) permit a better understanding of neurovascular and neurometabolic coupling; and (iii) provide a diagnostic tool for neurological disorders.

To capture quantitative glucose utilization information while providing temporal glucose utilization dynamics, we provide a novel PET imaging method. By infusing a radiotracer, such as FDG, at a constant rate, we access the temporal information that is not available through a bolus method. While a bolus FDG method is very powerful, the measurement of glucose utilization is the integral of these processes for 20-40 minutes following the injection of ¹⁸FDG as a bolus intravenously. This “snapshot” of glucose utilization thus has poor temporal resolution and does not contain intra-scan information about dynamic changes occurring during images acquisition. Our technique provides true dynamic FDG imaging with temporal resolution in the order of the minute.

The methods described herein allow for comparison of glucose utilization and oxygen consumption obtained simultaneously on similar time scales. This can improve the timing resolution of FDG-PET, and when combined with MRI provides a method for measuring true dynamic glucose utilization as a way to assess the differences in amplitude and temporal changes between fPET-FDG, fMRI BOLD, fMRI CBF, and CMRO₂ during cognitive tasks. The methods described herein will facilitate construction and testing of neurovascular and neurometabolic models.

Brain mapping of task-associated changes in hemodynamics and metabolism with positron emission tomography has been accomplished in the past by subtracting scans acquired during two distinct static states. Here we show that PET can provide truly dynamic information on neuronal energy metabolism. Using the widely available radiotracer, FDG, we show that quantitative glucose utilization changes during multiple visual stimuli can be determined from neuroimaging data acquired during FDG constant infusion in a single imaging experiment. Moreover, this functional PET (fPET) method can be accomplished simultaneously with fMRI (e.g., BOLD and ASL) and thus enables the first direct comparisons in time, space and magnitude of brain glucose utilization, hemodynamics and oxygen consumption.

These and other features, aspects, and advantages of the present invention will become better understood upon consideration of the following detailed description, drawings, and appended claims.

### BRIEF DESCRIPTION OF THE DRAWINGS

FIG. 1 is a schematic view of an emission tomography system suitable for use in accordance with the present invention.

FIG. 2 shows fPET-FDG activations maps. The top shows statistical activation maps observed for a single subject during the 10 minutes of full-field checkerboard presentation and the 5 minutes of left hemi-field checkerboard (z>1.5, non-corrected). The bottom shows the percentage signal change observed for a single subject during the 10 minutes full-field checkerboard and the 5 minutes left hemi-field checkerboard.

FIG. 3 shows an fPET-FDG experimental design. In (A), there is shown an fPET-FDG experimental design for a 90 minute visual paradigm alternating between a full-field checkerboard and left and right hemi-field checkerboard. In (B), there is shown an fPET-FDG signal in the occipital ROI (Regions of Interest), defined as the voxels activated during the full-field checkerboard paradigm, and in the a priori frontal lobe anatomical ROI. In (C), there is shown FDG signal change during the 90 minute experiment in the occipital ROI.

FIG. 4 shows a fixed-effect group analysis. In the top and bottom left, there is shown percentage signal change maps obtained for the fixed-effect group analysis (n=3) during the single 5 minute block of left hemi-field checkerboard, the single 5 minute block of right hemi-field checkerboard and the two blocks of 10 minutes full-field checkerboard. In the bottom right, there is shown percentage signal change values obtained in the a priori anatomical V1 ROI (see Amunts et al., Neuroimage 11, 66, January 2000) from the Jülich histological atlas for the fixed-effect group analysis (n=3) during the two blocks of 10 minutes full-field checkerboard, the single 5 minute block of left hemi-field checkerboard and the single 5 minute block of right hemi-field checkerboard.

FIG. 5 shows a comparison of the measurement of glucose utilization using the bolus SUV and the infusion slope method. In (A), there is shown an infusion slope map from a representative subject normalized to the average grey matter. In (B), there is shown a correlation between 3 subjects bolus SUV₄₀₋₇₀ and three subjects' infusion slope maps normalized to grey matter in 8 a priori anatomical ROIs. In (C), there is shown bolus SUV₄₀₋₇₀ map from a representative subject normalized to the average gray matter.

FIG. 6 shows a comparison between fPET-FDG, fMRI ASL and fMRI BOLD activations maps. At the top are statistical maps of the activations observed for a single subject during the two blocks of 10 minutes full-field checkerboard measured using fPET-FDG, fMRI ASL and fMRI BOLD (z>2, non-corrected); and at the bottom are percentage signal change observed for a single subject during the two blocks of 10 minutes of full-field checkerboard presentation measured using fPET-FDG, fMRI ASL and fMRI BOLD.

FIG. 7 shows fPET-FDG activations maps shown at different z-score thresholds. Statistical maps of the activations observed for a single subject during the two blocks of 10 minutes of full-field checkerboard presentation, the single 5 minute block of left hemi-field checkerboard and the single 5 minute block of right hemi-field checkerboard shown at three different z-score thresholds: z>1, non-corrected (top), z>1.5, non-corrected (middle), z>2, non-corrected (bottom).

FIG. 8 shows an fPET-FDG experimental design. A) shows fPET-FDG experimental design for a 90 minute experiment during visual paradigm alternating between a full checkerboard and left and right half checkerboard; B) shows fPET-FDG signal in the occipital ROI (red), defined as the voxels activated during the full checkerboard paradigm, and in the frontal ROI (black) in kBq/cc, the generalized linear model (GLM) used in the analysis in shown in black; and C) shows FDG signal in the occipital ROI after removing the baseline term, the black line represents the model we used to discriminate the slope changes for each stimulus.

FIG. 9 shows radioactivity concentration in the venous blood plasma and average time activity curve and derivative of the FDG signal in the whole brain. (A) shows venous blood was collected every 10 minutes in the arm of our 3 subjects during the 90 minute experiments. The interpolated average radioactivity concentration in the venous blood plasma of our 3 subjects (mean±std) is shown. (B) shows the normalized time activity curve in the whole brain average over the 3 subjects (mean±std) shows a linear increase during the entire 90 minutes experiment. (C) shows the derivative of the normalized time activity curve in the whole brain average over the 3 subjects (mean±std) shows that after around 30 minutes the derivative of the signal is stable.

FIG. 10 shows average CMR_{glu} map. Average CMR_{glu} map in μmol/100 g/min (scale from 0 to 0.6) across the 3 subjects. The CMR_{glu} maps were derived from the slope of the time-activity curve (TAC), normalized to venous blood plasma radioactivity concentration (in min⁻¹), multiplied by the glucose measurement (mmol/L) and divided by the lumped constant (0.89).

FIG. 11 shows a comparison of simulated and experimental responses with exponential fitting of the data. A) shows simulation of the effect of a brief change in k3 on the derivative of the FDG signal in the course of the infusion protocol. The red line represents the derivative of the FDG signal and the blue line represents the metabolized FDG. (B) shows normalized FDG signal change during the 10 minute full checkerboard for the 3 individual subjects (dashed lines) and the average response (red line). (C) shows average normalized FDG signal change during the 10 minute full checkerboard for the 3 individual subjects (red line) with an exponential fit for the increase in FDG signal after the beginning of the activation period (blue, R²=0.96, τ=4.9 min) and for the decrease in FDG signal after the end of the activation period (green, R²=0.98, τ=5.6 min). Exponential fitting was accomplished using GraphPad Prism® software (Prism6, GraphPad Software Inc., La Jolla, Calif., USA).

FIG. 12 shows fPET-FDG activations maps for a single subject. Statistical maps (T-score>6) of the activations observed for a single subject during the two blocks of 10 minutes full-field checkerboard presentation, the single 5 minute block of left hemi-field checkerboard, and the single 5 minute block of right hemi-field checkerboard.

FIG. 13 shows fixed-effects group analysis. (top and bottom left) Percent signal change maps obtained for the fixed-effects group analysis (n=3) during the single 5 minute block of left hemi-field checkerboard, the single 5 minute block of right hemi-field checkerboard, and the two blocks of 10 minutes full-field checkerboard; (bottom right) Percent signal change values obtained in the a priori anatomical V1 ROI from the Jülich histological atlas for the fixed-effect group analysis (n=3) during the two blocks of 10 minutes full-field checkerboard, the single 5 minute block of left hemi-field checkerboard and the single 5 minute block of right hemi-field checkerboard.

FIG. 14 shows non-human primate experiment. The red line shows a linear increase of FDG signal in the grey matter of one anesthetized baboon during the 50 minute constant infusion. A hypercapnic challenge (7% CO₂) was administered between 30 and 40 minutes and ASL was acquired around the challenge to measures changes in CBF. The blue line shows an increase in average percent signal change in CBF measured in the grey matter using ASL during the hypercapnic challenge, followed by a decrease in CBF after the end of the hypercapnic challenge.

### DETAILED DESCRIPTION OF THE INVENTION

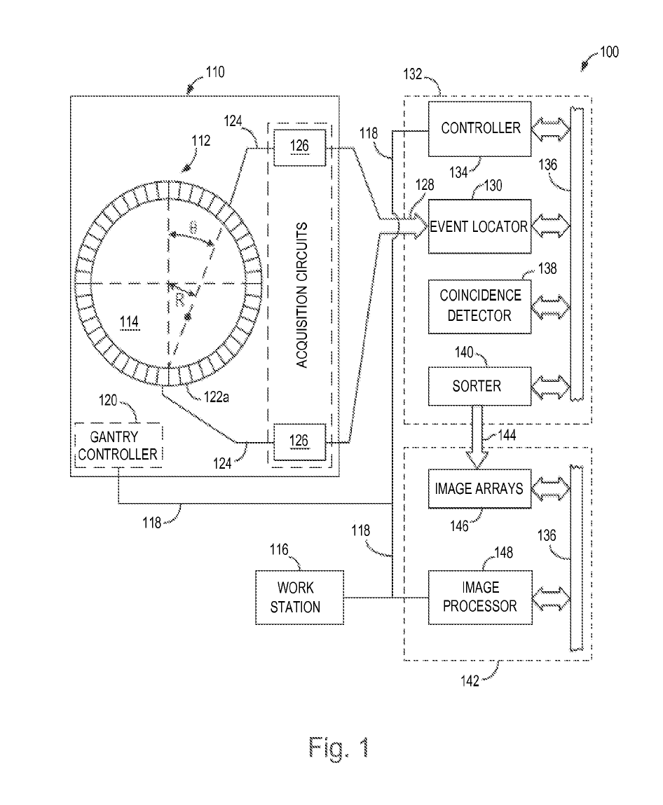

Referring to FIG. 1, a PET system 100 that can be used in the method of present invention includes an imaging hardware system 110 that includes a detector ring assembly 112 about a central axis, or bore 114. An operator work station 116 including a commercially-available processor running a commercially-available operating system communicates through a communications link 118 with a gantry controller 120 to control operation of the imaging hardware system 110.

The detector ring assembly 112 is formed of a multitude of radiation detector units 122 that produce a signal responsive to detection of a photon on communications line 124 when an event occurs. A set of acquisition circuits 126 receive the signals and produce signals indicating the event coordinates (x, y) and the total energy associated with the photons that caused the event. These signals are sent through a cable 128 to an event locator circuit 130. Each acquisition circuit 126 also produces an event detection pulse that indicates the exact moment the interaction took place. Other systems utilize sophisticated digital electronics that can also obtain this information regarding the precise instant in which the event occurred from the same signals used to obtain energy and event coordinates.

The event locator circuits 130 in some implementations, form part of a data acquisition processing system 132 that periodically samples the signals produced by the acquisition circuits 126. The data acquisition processing system 132 includes a general controller 134 that controls communications on a backplane bus 136 and on the general communications network 118. The event locator circuits 130 assemble the information regarding each valid event into a set of numbers that indicate precisely when the event took place and the position in which the event was detected. This event data packet is conveyed to a coincidence detector 138 that is also part of the data acquisition processing system 132.

The coincidence detector 138 accepts the event data packets from the event locator circuit 130 and determines if any two of them are in coincidence. Coincidence is determined by a number of factors. First, the time markers in each event data packet must be within a predetermined time window, for example, 0.5 nanoseconds or even down to picoseconds. Second, the locations indicated by the two event data packets must lie on a straight line that passes through the field of view in the scanner bore 114. Events that cannot be paired are discarded from consideration by the coincidence detector 138, but coincident event pairs are located and recorded as a coincidence data packet. These coincidence data packets are provided to a sorter 140. The function of the sorter in many traditional PET imaging systems is to receive the coincidence data packets and generate memory addresses from the coincidence data packets for the efficient storage of the coincidence data. In that context, the set of all projection rays that point in the same direction (θ) and pass through the scanner's field of view (FOV) is a complete projection, or “view”. The distance (R) between a particular projection ray and the center of the FOV locates that projection ray within the FOV. The sorter 140 counts all of the events that occur on a given projection ray (R, θ) during the scan by sorting out the coincidence data packets that indicate an event at the two detectors lying on this projection ray. The coincidence counts are organized, for example, as a set of two-dimensional arrays, one for each axial image plane, and each having as one of its dimensions the projection angle θ and the other dimension the distance R. This θ by R map of the measured events is call a histogram or, more commonly, a sinogram array. It is these sinograms that are processed to reconstruct images that indicate the number of events that took place at each image pixel location during the scan. The sorter 140 counts all events occurring along each projection ray (R, θ) and organizes them into an image data array.

The sorter 140 provides image datasets to an image processing/reconstruction system 142, for example, by way of a communications link 144 to be stored in an image array 146. The image arrays 146 hold the respective datasets for access by an image processor 148 that reconstructs images. The image processing/reconstruction system 142 may communicate with and/or be integrated with the work station 116 or other remote work stations.

In one embodiment, the invention provides an emission tomography system for acquiring a series of medical images of a subject during an imaging process using a radiotracer. The system includes a plurality of detectors configured to be arranged about the subject to acquire gamma rays emitted from the subject over a time period relative to an administration of the radiotracer to the subject and communicate signals corresponding to acquired gamma rays. A “subject” is a mammal, preferably a human. The system also includes a stimulus system configured to track a stimulus associated with the subject over the time period and communicate a stimulus feedback. The system further includes a data processing system configured to receive the signals from the plurality of detectors and the stimulus feedback from the stimulus system and correlate the signals with information about the stimulus associated with the subject. The system also includes a reconstruction system configured to receive correlated signals and information about the stimulus from the data processing system and reconstruct therefrom a series of medical images of the subject showing a physiological response of the subject to the stimulus over the time period. Reconstruction can be done in real time. In one version of the system, a second series of medical images is concurrently acquired using a magnetic resonance imaging device.

The stimulus used in the stimulus system can be one or more of a visual stimulus, an auditory stimulus, and a mechanical stimulus (which includes, without limitation, motor function). In one non-limiting example, a visual stimulus comprising a counterphase flickering “checkerboard” pattern is used. The stimulus may include at least two of a visual stimulus, an auditory stimulus, and a mechanical stimulus. Thus, multiple tasks can be modeled. For example, concurrent use of a visual and motor stimulus may show different regions of the cortex lighting up on the acquired medical images. The stimulus can be a sensory stimulus initially targeted to one or more senses of the subject, such as sight, hearing, taste, smell, touch, and proprioception. The stimulus system preferably uses a behavioral paradigm, i.e., a normal physiological stimulus, not a drug.

In one version of this embodiment, the stimulus is a sensory stimulus directed to the brain of the subject, and the detectors are arranged to acquire gamma rays emitted from the brain of the subject, and the reconstruction system receives correlated signals and information about the stimulus from the data processing system and reconstructs therefrom a series of medical images of the brain of the subject. The physiological response of the subject to the stimulus includes activation of portions of a brain of the subject in response to the stimulus.

In one version of this embodiment, the radiotracer comprises a positron emitter. In one version, the radiotracer comprises at least one of ¹¹C, ¹³N, ¹⁵O, ¹⁸F, ⁶⁴Cu, ⁶⁸Ga, ⁷⁵Br, ⁷⁶Br, ⁸²Rb, ⁸⁹Zr, ¹²¹I, and ¹²⁴I. Preferably, the radiotracer includes ¹⁸F-fluorodeoxyglucose (FDG). The system can include an administration system configured to deliver the radiotracer to the subject at a controlled rate. The system can include an administration system configured to deliver the radiotracer to the subject at a constant rate. In one non-limiting example, the administration system is configured to deliver the radiotracer to the subject at a constant rate in the range of 0.001 to 0.1 milliliters per second, or 0.001 to 0.05 milliliters per second, or 0.005 to 0.02 milliliters per second, or about 0.01 milliliters per second for a time period in the range of 15 to 240 minutes, or 30 to 180 minutes, or 60 to 120 minutes.

In one version of this embodiment, the reconstruction system is configured to derive a parametric map from time series data. In another version of this embodiment, the reconstruction system is configured to derive cerebral metabolic rate of glucose (CMR_{glu}) from time series data.

In another embodiment, the invention provides a method of dynamically imaging a subject over a set time period by emission tomography. The method includes the steps of (a) administering a radiotracer to the subject continuously over the set time period; (b) directing one or more tracked stimuli to the subject during the set time period; (c) using a plurality of detectors to detect gamma rays emitted from the subject and to communicate signals corresponding to the detected gamma rays over the set time period; (d) correlating the signals with information about the tracked stimuli directed to the subject; and (e) reconstructing from this correlation a series of medical images of a region of interest of the subject.

In one version of this embodiment, step (b) comprises directing one or more tracked sensory stimuli to the brain of the subject. The physiological response of the subject to the stimulus can include activation of portions of a brain of the subject in response to the stimulus, and the series of medical images can show a physiological response to the tracked stimuli over the time period. The tracked stimuli can include at least one of a visual stimulus, an auditory stimulus, and a mechanical stimulus. The tracked stimuli can include at least two of a visual stimulus, an auditory stimulus, and a mechanical stimulus. The tracked stimuli can be directed to the brain of the subject and are initially targeted to one or more of the senses of the subject, such as sight, hearing, taste, smell, touch, and proprioception.

In one version of the method, the radiotracer comprises a positron emitter. In one version, the radiotracer comprises at least one of ¹¹C, ¹³N, ¹⁵O, ¹⁸F, ⁶⁴Cu, ⁶⁸Ga, ⁷⁵Br, ⁷⁶Br, ⁸²Rb, ⁸⁹Zr, ¹²¹I, and ¹²⁴I. Preferably, the radiotracer includes ¹⁸F-fluorodeoxyglucose (FDG). The radiotracer can be administered intravenously to the subject at a controlled rate. The radiotracer can be administered intravenously to the subject at a constant rate. In one non-limiting example, the constant rate in the range of 0.001 to 0.1 milliliters per second, or 0.001 to 0.05 milliliters per second, or 0.005 to 0.02 milliliters per second, or about 0.01 milliliters per second for a time period in the range of 15 to 240 minutes, or 30 to 180 minutes, or 60 to 120 minutes.

In one version of the method, a parametric map is derived from time series data. In another version of the method, cerebral metabolic rate of glucose (CMR_{glu}) is derived from time series data. In one version of the method, a second series of medical images is concurrently acquired using magnetic resonance imaging.

The invention provides a novel PET approach that images glucose metabolism dynamically using the widely available 2-[¹⁸F]-fluoro-deoxyglucose (FDG) during time varying neuronal tasks. We have termed the new method functional PET (fPET) and have demonstrated with validation studies that fPET-FDG provides dynamic information in a similar manner to fMRI by BOLD (blood oxygen level dependent) signal or ASL (arterial spin labeling) measurements of CBF (cerebral blood flow). Performed on combined PET/MRI devices, investigators thus now have the ability to simultaneously measure dynamic molecular events along with physiological events during changing brain states.

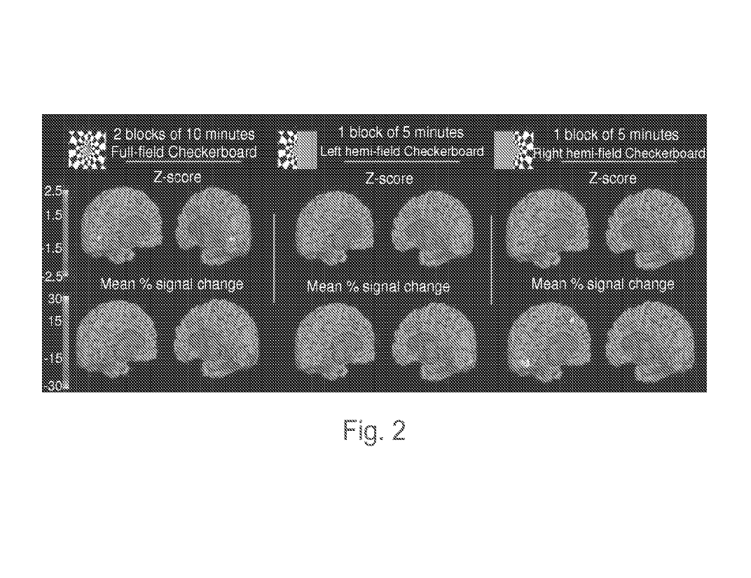

The visual system, a standard and robust test system for new neuroimaging techniques, was used as a non-limiting example to demonstrate that PET can operate in a temporally dynamic mode. To interrogate dynamic metabolic information, a 90-minute constant FDG infusion paradigm was used (0.01 ml/s, 5 mCi total calculated to the beginning of infusion), during which we periodically presented a conventional visual stimulus consisting of an 8-Hz counterphase flickering “checkerboard” pattern as shown in FIG. 2A. The stimulus was projected on a screen mounted at the end of the magnet bore, and subjects (n=3) viewed the stimulus through a mirror. We used three different stimuli: two full-field checkerboard stimulus periods for a duration of 10 minutes each, and two hemi-field checkerboard stimulus periods (right and left) for a duration of 5 minutes each (FIG. 2A). This design allowed us to confirm that the observed metabolic signal changes were specific to the stimuli, and also that multiple stimuli (four in this case) can be measured dynamically during a single scan of a single subject. In addition to our dynamic analysis, the fPET-FDG data were also binned into 90 one-minute frames using previously published methods (see, Catana et al., J Nucl Med 51, 1431, September, 2010; and Catana et al., J Nucl Med 52, 154, January 2011). During the fPET-FDG scan, functional MR data were simultaneously collected using a Siemens BrainPET scanner. BOLD data were collected prior to and during the first 10 minute full-field checkerboard and the two hemi-field checkerboard stimuli; and ASL data were collected prior to and during the second 10 minute full-field checkerboard stimulus.

FDG was delivered at a constant rate during our fPET studies. At steady state, the rate of FDG utilization in a given voxel is proportional to the slope of the radioactivity accumulated in that voxel. As shown in Example 1 below, during steady state the slope of the voxelwise FDG uptake from fPET can be represented as: slope=(CMRglu*LC*Cp)/Cₐ^{o} where LC is the lumped constant which converts FDG metabolic rate to glucose metabolic rate, Cₐ^{o} is a measurement of plasma glucose (see, Reivich et al., Circ Res 44, 127, January 1979; and Phelps et al., Ann Neurol 6, 371, November 1979), and Cₚ is the plasma radioactivity concentration (which is controlled through constant infusion). The slope is an approximation for (K₁k₃)/(k₂+k₃) scaled by Cₚ and is thus analogous to Kᵢ (the influx rate constant) determined by a linear approximation of a bolus FDG injection by Patlak analysis (see, Patlak et al., J Cereb Blood Flow Metab 3, 1, Mar. 1983; and Lucignani et al., J Nucl Med 34, 360, March 1993). Changes in slope are thus proportional to changes in CMR_{glu}. By taking the derivative of the fPET-FDG uptake time course estimated using pairwise subtraction of the 90 one minute frames to form a relative index of uptake rate, we generated a time series akin to a BOLD fMRI signal to represent change in CMR_{glu}. We then used fMRI software, FSL's (FMRIB Software Library) FEAT (see Jenkinson et al., Neuroimage 62, 782, Aug. 15, 2012) to process the fPET-FDG data using a generalized linear model (GLM) with 3 different explanatory variables (EV): (i) 2 blocks of the 10 minutes full-field checkerboard paradigm (EV1), (ii) a 5 minute block of left hemi-field checkerboard (EV2) and (iii) a 5 minute block of right hemi-field checkerboard (EV3). The fPET-FDG activation maps for the three contrasts of interest for a single subject are shown in FIG. 2. Confirmation of functional specificity is inherent in our analyses given that both hemispheres of the visual system respond equally to the full-field checkerboard whereas the contralateral hemisphere has a higher response to a hemi-field checkerboard.

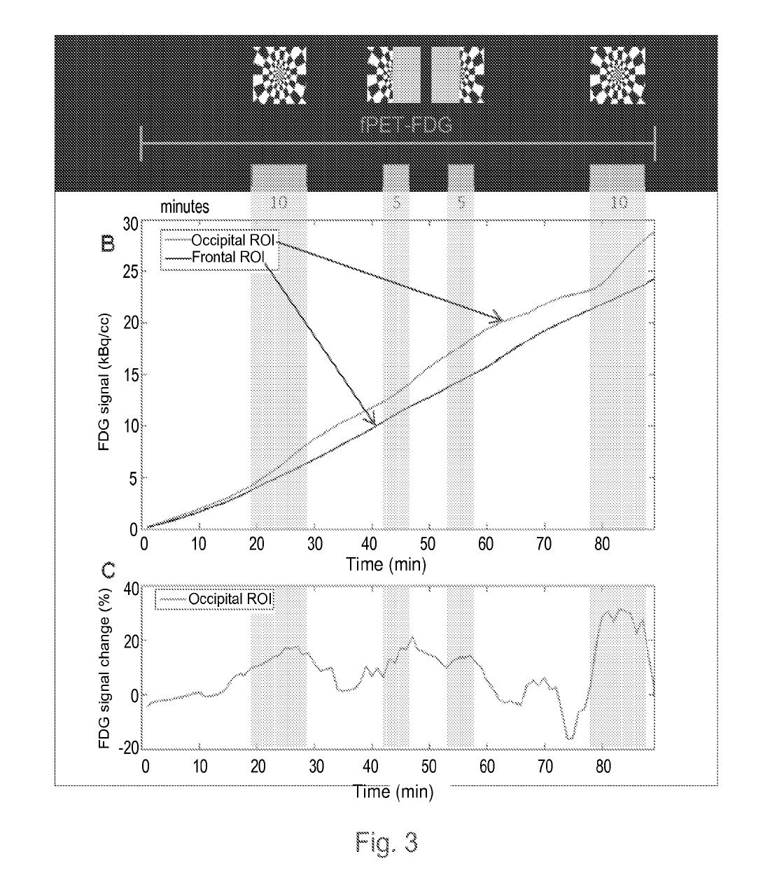

Using the activated voxels to define a post-hoc volume-of-interest, an individual subject's time-activity curve (TAC) for visual stimulation was created and compared to a volume-of-interest that did not respond to the visual stimulus (the individual subject's frontal cortex). Changes in glucose utilization are easily observed as changes in the TAC slope (FDG utilization rate) during visual stimulus in the activated voxels (occipital region) but not in the frontal cortex (see FIG. 3B). These same data are presented as a derivative (through pair-wise subtraction) in FIG. 3C.

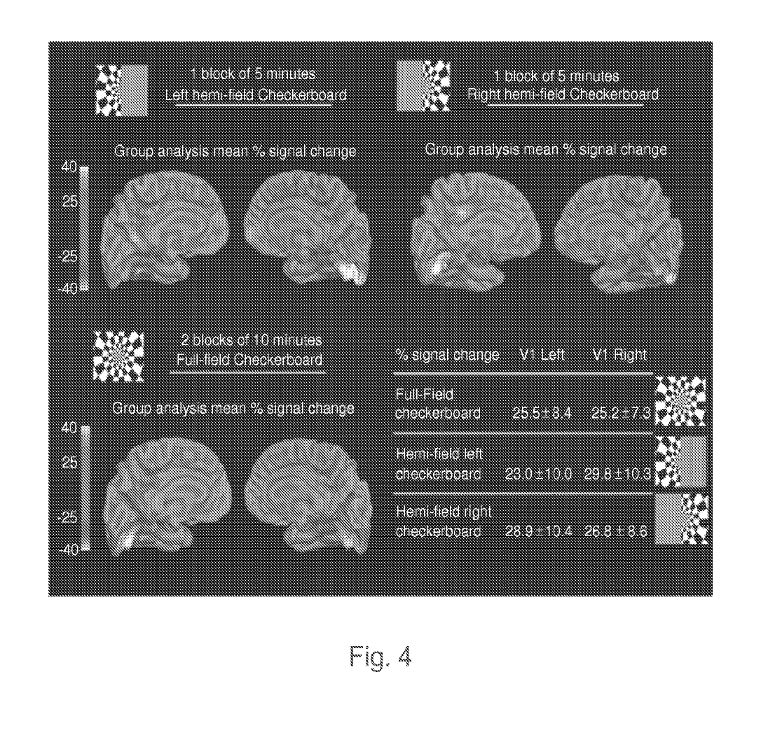

Group analysis of the fPET-FDG data set from three subjects was accomplished using fixed-effect analysis (see FIG. 4) to determine the average percent change in glucose utilization during each visual stimulus (i.e. left-, right- and full-checkerboard) compared to baseline. The mean percent increase in glucose utilization derived from fPET-FDG for our three subjects was 25% for the full-field checkerboard, 26% for the left hemi-field checkerboard and 28% for the right hemi-field checkerboard (see FIG. 4). The contralateral increase was higher for both half-checkerboard stimuli and the absolute changes in FDG utilization as measured by fPET-FDG are consistent with previous studies measuring single response in a two-scan paradigm (see, Newberg et al., Neuroimage 28, 500, Nov. 1, 2005).

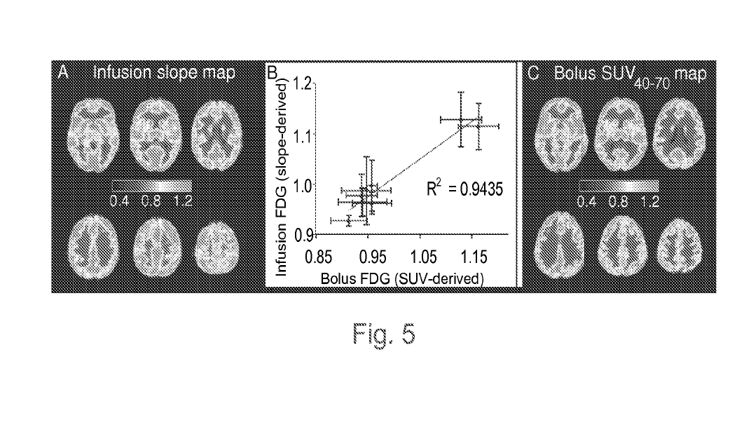

The baseline fPET-FDG voxel-wise slope provides relative measure of cerebral rate of glucose metabolism (CMR_{glu}), which has previously been correlated with standardized uptake values (SUVs) (see H. Suhonen-Polvi et al., J Nucl Med 36, 1249, July 1995; and Yamaji et al., Clin Nucl Med 25, 11, January 2000). We empirically demonstrated a relationship between SUV and fPET-FDG index of relative CMR_{glu}. As with the visual stimulus experiments above, we administered FDG at a constant infusion rate of 0.01 ml/s for 90 minutes to 3 healthy subjects (1 female/2 males, mean age 32±2) and compared the results to those obtained with traditional bolus injections of FDG (5 mCi) during rest for three additional healthy subjects (1 female/2 male, mean age 29±3). Using the infusion data set, we created a ‘slope’ image by voxel-wise linear regression of each voxel's time-activity curve for each subject. For comparison, a voxel-wise SUV image was created for each subject from the bolus-derived data (average FDG uptake 40-70 minutes, post injection, normalized to injected dose and subject mass). Regions of interest were defined in a subject-specific manner using FreeSurfer (see, B. Fischl, Neuroimage 62, 774, Aug. 15, 2012) processing of the concurrently acquired MRI anatomical sequence (multi-echo MPRAG). Eight subject-specific a priori anatomical regions of the brain (frontal, occipital, parietal, and temporal lobe, caudate, cerebellum, insula, putamen) were averaged for each of the two groups and the mean results of the bolus SUV and infusion slope were normalized to the mean gray matter values for each subject (see FIG. 5). The correlation between the slope-derived and the SUV-derived images is significant with a Pearson's correlation coefficient of 0.94 (p<0.001). Note that imperfect correlation is expected because of actual biological variability between subjects.

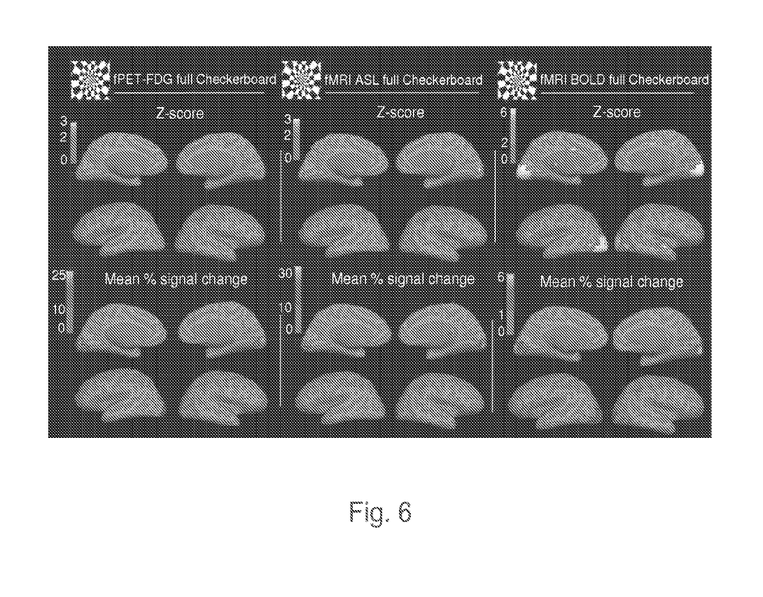

These data demonstrate that signal changes in fPET-FDG are directly related to changes in CMR_{glu}, and simultaneously measured flow data provide a means to estimate sources of error in CMR_{glu} (see, Reivich et al., Res Publ Assoc Res Nery Ment Dis 63, 105, 1985; and Hasselbalch et al., J Cereb Blood Flow Metab 21, 995, August 2001). As expected, CBF increased during visual stimulation (see FIG. 6). fPET-FDG should not be influenced by CBF changes that occurred during the activation paradigm, given that FDG has very low extraction fraction, only 4%, and as such utilization is flow independent except under extreme conditions of hypoglycemia (see, Reivich et al., Res Publ Assoc Res Nery Ment Dis 63, 105, 1985; and Schuier et al., J Cereb Blood Flow Metab 10, 765, November 1990). Similarly, small changes in CBV also occur during visual activation (see, M. E. Phelps et al., Ann Neurol 6, 371, November 1979) and will contribute to the fPET-FDG signal but minimally given that each image voxel on average consists of a <5% blood component. By measuring blood radioactivity during the infusion protocol (average 4 kBq/cc whole blood), we determined that a 9% change in CBV, as estimated from the measured change in CBF (see, R. L. Grubb, Jr. et al., Stroke 5, 630, September-October 1974), would increase the fPET-FDG signal only by 0.5%. Such a change would make a negligible contribution to our current fPET signal, and could be independently modeled in situations where greater effects might be considered.

fPET-FDG provides a simple and quantitative means to observe brain glucose utilization changes dynamically in a single imaging session. The method is an operationally straightforward “repurposing” of this fundamental PET index of metabolism, and can be performed on any commercial PET scanner (such as illustrated in FIG. 1) using a widely available and inexpensive radiotracer. Looking forward, the complementary nature of fPET-FDG to fMRI capitalizes on the emerging technology of hybrid MR-PET scanners. For example, fPET-FDG, combined with rapidly evolving quantitative fMRI methods, will allow us to simultaneously measure dynamic changes in glucose utilization, hemodynamics and oxygen consumption, addressing vital questions about neuronal and neurovascular relationships across tasks and disease states. Indeed many questions remain about normal human brain physiology (e.g., why and when are changes in CMRO₂ and CMR_{glu} non-stochastic?), and regarding dysfunctions in metabolic/hemodynamic coupling that occur in many diseases.

More broadly, our data point towards the capacity of fPET to dynamically image molecular events with the exquisite sensitivity of PET during multi-task challenges. The molecular specificity of a wide range of metabolic and neuroreceptor targeted PET tracers undoubtedly expand fPET beyond measurements of glucose utilization dynamics (see, C. Y. Sander et al., Proc Natl Acad Sci USA 110, 11169, Jul. 2, 2013). We anticipate that fPET will be a modular imaging technique that is extensible to both existing radiotracers and those to be designed specifically for fPET, and one which will provide new temporal information on multifaceted neuronal molecular events, which heretofore have been measured as static ‘state’ functions or an accumulated value. While the temporal resolution of such methods will depend on a number of factors, both technical and biological, the principle of dynamic imaging of metabolic or neuroreceptor status will provide an important new dimension to efforts to quantify brain structure/function relationships.

The invention is further illustrated in the following Examples which are presented for purposes of illustration and not of limitation.

### EXAMPLES

### Example 1

The imaging studies were performed on a 3-T Tim MAGNETOM Trio MR scanner (Siemens Healthcare, Inc) modified to support the BrainPET (Siemens), an MR-compatible brain-dedicated PET scanner prototype. The BrainPET, which uses magnetic-field-insensitive avalanche photodiodes in combination with lutetium oxyorthosilicate crystals as photodetectors, has a transaxial/axial field of view of 32/19.125 cm. Three-dimensional (3D) coincidence event data are collected with a maximum ring difference of 67 and stored in a list-mode format. For reconstruction, the list mode files are sorted into line-of-response space and further compressed into sinogram space. The PET data were reconstructed with a standard 3D ordinary Poisson ordered-subset expectation maximization algorithm using both prompt and variance-reduced random coincidence events as well as normalization, scatter, and attenuation sinograms. The attenuation sinograms were derived from dual-echo ultrashort echo time (DUTE) MR images using a novel algorithm incorporating corrections from an individual's structural (MPRAGE) scan. The data were reconstructed with a voxel size of 1.25-mm isotropic into a volume consisting of 153 transverse slices of 256×256 pixels. The volumes are smoothed using a 3D filter with a 3-mm isotropic Gaussian kernel.

Magnetic resonance imaging was performed using two concentric head coils: an outer circularly polarized transmit-receive coil and an inner 8-channel receive-only coil specially designed for the BrainPET with considerations for their 511-keV photon attenuation properties.

PET Protocol:

FDG was administered in saline intravenously at a constant infusion rate of 0.01 ml/s for 90 minutes to healthy subjects (1 female/2 males, mean age 32±2) using a Medrad® Spectra Solaris syringe pump. In a separate experiment, FDG was administered as a traditional bolus injection (5 mCi administered in 5 mL saline over ˜30 seconds) during rest for 3 additional healthy subjects (1 female/2 males, mean age 29±3). Venous blood samples were collected in all subjects during scanning, then centrifuged to obtain plasma, and aliquoted in a gamma counter that had been precalibrated to the PET scanner to measure the venous activity during the experiment. The fPET-FDG data were binned into 90 one-minute frames. The reconstructed volume was down-sampled to 76 slices with 128×128 voxels (2.5×2.5×2.5 mm³) with one minute time frames. For bolus FDG comparison studies, data were processed as a single frame (40-70 minutes post injection) and analogously down-sampled. Standardized Uptake Values (SUVs) were normalized to gray matter uptake for each subject.

MRI Acquisition:

Anatomical studies consisted in a high-resolution T1-weighting acquired using multi-echo MPRAGE (TR=2530 ms, TE₁/TE₂/TE₃/TE₄=1.64/3.5/5.36/7.22 ms, T1=1200 ms, flip angle=7°, and 1 mm isotropic) and a dual ultra-short echo sequence with TR=200 ms, TE₁/TE₂=0.07/2.24 ms, flip angle=10°, and 1.67 mm isotropic resolution, run to derive the PET attenuation map. These sequences were acquired parallel to the anterior commissure-posterior commissure (AC-PC) plane. BOLD imaging consisted in a T₂*-weighted GRE Echo Planar Imaging (EPI) acquisition (TR/TE=3000/30 ms, matrix=72×72, field of view=21.6×21.6 cm (3 mm isotropic resolution), and 47 slices without gaps) with whole-brain coverage. BOLD data were acquired before, during and after the first full-field checkerboard paradigm and around the two hemi-field checkerboard paradigms with 5 minutes of baseline before and after each paradigm. Perfusion imaging consisted of 20 minutes (5 minutes of baseline before and after the 10 minute full-field paradigm) of a pseudo-continuous arterial spin labeling (pCASL) technique acquisition before and during the second full-field checkerboard (gradient-echo EPI with TR/TE₁/TE₂=4000/10/30 ms, labeling duration=1.6 seconds, post-labeling delay=1 seconds, 3.4×3.4×6 mm spatial resolution, GRAPPA (R=2) acceleration, and 7/8 partial Fourier).

### Data Processing

fPET-FDG Processing: Subject Level (See FIGS. 2 & 3):

Pairwise subtraction of the 90 images was accomplished with JIP (http://www.nitrc.org/projects/jip/). For example, frame 90-frame 89 was assigned as time point 90; frame 89-frame 88 was assigned as time point 89, etc. This process generated a time series related to the derivative of radioactivity accumulating in each voxel. The program FEAT v.6.0 from FSL's analysis package (see J. R. Polimeni et al., Neuroimage 52, 1334, Oct. 1, 2010) was used without pre-processing other than the application of a 12 mm Gaussian smoothing kernel to analyze the PET time series using a general linear model (GLM) consisting of 3 different explanatory variables (EV) as outlined above. Data from a single subject are shown at z>1.5 in FIG. 2. The activated voxels (z>1.5) from the same subject were used to generate a region-of-interest, which was projected onto the original PET volume to generate the time-activity curve (TAC) in FIG. 3b. The frontal cortex TAC was generated by projecting the anatomical subject-specific ROI from the FreeSurfer cortical surface reconstruction onto the PET data. Percent signal change from the activated voxels (FIG. 3C) was derived from GLM analysis in FSL.

fPET-FDG Processing: Subject Level (See FIG. 6):

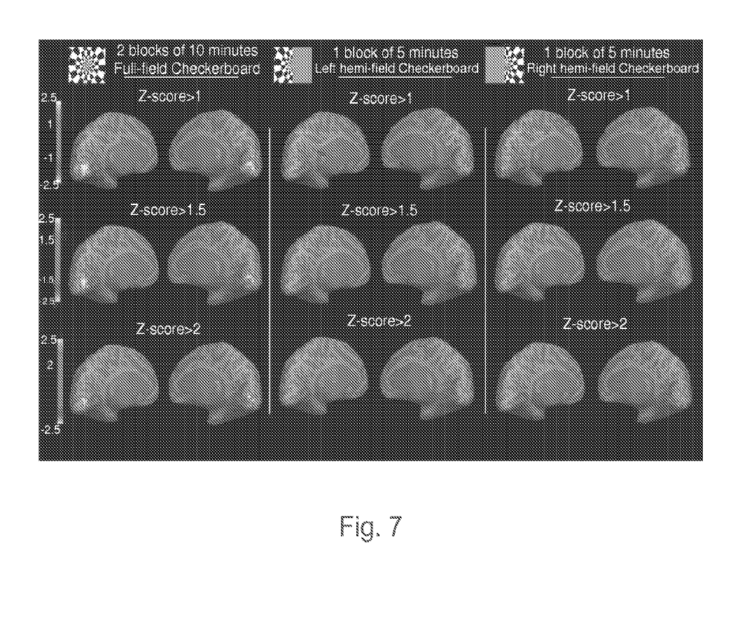

Comparisons between fPET-FDG (as detailed above), fMRI ASL and fMRI BOLD activations maps during the 10 minute full-field checkerboard presentation for the same subject are shown in FIG. 6. ASL and BOLD were processed using the standard FSL software. MR data were motion corrected with FSL using MCFLIRT, smoothed with a 6 mm Gaussian kernel; relative CBF maps were calculated via control/tag subtraction performed on the ASL data. The z-scores maps were thresholded at z>2 (p<0.05, non-corrected) and the percentage signal change values observed using fPET-FDG, fMRI ASL and fMRI BOLD were calculated are shown in FIG. 6. The data from these measurements are visualized on independent scales since the percent signal change and z-scores vary greatly between fPET-FDG, CBF and BOLD signal. For comparison, fPET-FDG activations maps for one single subject at different z-scores thresholds are shown on FIG. 7.

fPET-FDG Processing: Fixed-Effect Group Analysis (See FIG. 4):

In order to concatenate the time series data for our three subjects, we normalized each subject's PET volume (from pairwise subtraction) to the MNI brain atlas template using the MPRAGE image volume for co-registration. This concatenated time series of 90*3 images was processed using FSL FEAT software as a fixed-effect group analysis. From this analysis we obtained the statistical significance of the effect and the percent signal change for all voxel within the group (n=3). The percent change for the group for the 3 EVs (full-field, hemi-field left, hemi-field right checkerboard stimulus) at the group level on FIG. 4. Mean percent change±standard deviation for the left and right V1 a priori ROIs (defined anatomically using the Jülich histological atlas) were extracted from FSL for each of the 3 contrasts (see the Table within FIG. 4).

Comparison Between Infusion Slope Maps and Bolus SUV (See FIG. 5):

The SUV maps were calculated as the ratio of each voxel's radioactivity during the last set of time points (40 to 70 minutes) and the injected activity at the time of injection divided by the body weight (FIG. 5A). The infusion slope map was calculated voxel-by-voxel using a nonlinear regression of the voxel-wise time activity curve signal FDG using custom Matlab® software (FIG. 5C). The regions of interest were defined in a subject-specific manner using the FreeSurfer software package (see Kwong et al., Proc Natl Acad Sci USA 89, 5675, Jun. 15, 1992) which automatically generated cortical surface models and a brain region parcellation for each volunteer from the T₁-weighted MPRAGE image volume acquired in the same experimental session. Eight subject-specific regions of the brain (frontal, occipital, parietal, and temporal lobe, caudate, cerebellum, insula, putamen) were each averaged for the SUV and slope groups and the mean results of the bolus SUV and infusion slope were normalized to the mean gray matter values for each subject (see FIG. 5B).

Relationship of fPET-FDG to CMR_{glu}

Classic analysis of FDG kinetics is accomplished using an irreversible two-tissue compartment model. FDG is trapped in the tissue after initial phosphorylation and thus k₄ is small and approximated as null for the duration of experiment. Using this compartment model, the following partial differential equations can be written to describe the system:

$$\begin{matrix}\frac{d {C}_{p} ( t )}{d t} = {k}_{2} {C}_{f} - {k}_{1} {C}_{p} & ( 1 ) \\ \frac{d {C}_{f} ( t )}{d t} = {k}_{1} {C}_{p} - ( {k}_{2} + {k}_{3} ) {C}_{f} & ( 2 ) \\ \frac{d {C}_{m} ( t )}{d t} = {k}_{3} {C}_{f} & ( 3 )\end{matrix}$$

Cₚ is the plasma compartment; C_{f} is a tissue compartment representing free FDG; and Cₘ is the compartment representing metabolized (phosphorylated) FDG.

### Model Simplification During Rest:

Given that FDG was infused at a constant rate during fPET studies, we can assume that after initial equilibration period, the freely available FDG for metabolism at rest is constant, hence:

$$\frac{d {C}_{f} ( t )}{d t} = {k}_{1} {C}_{p} - ( {k}_{2} + {k}_{3} ) {C}_{f} = 0 \qquad ( 4 )$$

By rearranging to solve for C_{f} from Eqn. 4 and substituting into Eqn. 3:

$$\frac{d {C}_{m} ( t )}{d t} = {C}_{p} \frac{{k}_{1} {k}_{3}}{{k}_{2} + {k}_{3}} \qquad ( 5 )$$

Eqn. 5 can be corrected to CMR_{glu} using the lumped constant, which converts FDG metabolic rate to glucose and Cₐ^{o}, which is a measurement of plasma glucose. Thus,

$$CMRglu = \frac{{C}_{a}^{°}}{( LC ) ( {C}_{p} )} ( \frac{d {C}_{m} ( t )}{d t} ) \qquad ( 6 )$$

The PET measurement is related to the total concentration of the two-tissue compartments, hence it follows that:

$$slope ≡ \frac{d {C}_{r} ( t )}{d t} = \frac{d {C}_{f} ( t )}{d t} + \frac{d {C}_{m} ( t )}{d t} \qquad ( 7 )$$

$$\frac{d {C}_{f} ( t )}{d t} = 0 , \frac{d {C}_{r} ( t )}{d t} = \frac{d {C}_{m} ( t )}{d t} .$$

When Thus, the slope observed by fPET-FDG is equal to dCₘ/dt and as seen in Eqn. 6 proportional to CMR_{glu}.

### Model Assumptions During Task:

During initiation of a task, compartment concentrations change such C_{f} may not be constant. If we assume that after a transient period that is short relative to the task, we reach a new steady state (different from rest) where

$$\frac{d {C}_{f} ( t )}{d t} = 0 ,$$

we can again approximate average CMR_{glu} during the task using slope. The % change between slopes measured during two states (rest and task) is thus equal to % change in CMR_{glu}, when Cₐ^{o} and Cₚ are constant.

It is worth noting that lumped constant values vary across studies (0.52 to 0.89) and that absolute measurements have additional ambiguities and assumptions. See, Graham et al., “The FDG lumped constant in normal human brain”, J Nucl Med. 2002; 43(9):1157-66.

### Example 2

Example 2 is a study similar to Example 1 and provides further refinements to the technique of Example 1.

### Overview of Example 2

Glucose is the principal source of energy for the brain and yet the dynamic response of glucose utilization to changes in brain activity is still not fully understood. Positron emission tomography (PET) allows quantitative measurement of glucose metabolism using 2-[¹⁸F]-fluorodeoxyglucose (FDG). However, FDG PET in its current form provides an integral (or average) of glucose consumption over tens of minutes and lacks the temporal information to capture physiological alterations associated with changes in brain activity induced by tasks or drug challenges. Traditionally, changes in glucose utilization are inferred by comparing two separate scans, which significantly limits the utility of the method. In this Example, we report a novel method to track changes in FDG metabolism dynamically, with higher temporal resolution than exists to date and within a single session. Using a constant infusion of FDG, we demonstrate that our technique (termed fPET-FDG) can be used in an analysis pipeline similar to fMRI to define within-session differential metabolic responses. We use visual stimulation to demonstrate the feasibility of this method. This new method has a great potential to be used in research protocols and clinical settings since fPET-FDG imaging can be performed with most PET scanners and data acquisition and analysis is straightforward. fPET-FDG is a highly complementary technique to MRI and provides a rich new way to observe functional changes in brain metabolism.

### Introduction

As noted above, although the brain represents only 2% of the body weight, it receives 15% of the cardiac output, 20% of total body oxygen consumption, and 25% of total body glucose utilization. Glucose utilization in the human brain, both at rest and during cognitive tasks, has been studied over the past 40 years using 2-[¹⁸F]-fluoro-deoxyglucose (FDG) with positron emission tomography (PET) (Phelps et al., 1979; Reivich et al., 1979). However, the measurement of glucose utilization represents an integral of neuronal processes during 20-40 minutes following an intravenous bolus injection of FDG. This “snapshot” of glucose utilization is like a picture with a very long exposure, with poor temporal resolution and without intra-scan information about dynamic changes occurring during this extended image acquisition. Inferences of changes in glucose metabolism in response to stimuli or tasks are obtained with state-contrast experiments. The bolus FDG method, widely used clinically, can provide a quantitative measurement of the basal cerebral metabolic rate of glucose (CMR_{glu}) and is a very powerful way to characterize functional metabolic responses to stimuli that are presumed to sustain a constant state, including visual, auditory or cognitive tasks, and even drug administration (Gould et al., 2012; Kushner et al., 1988; Molina et al., 2009; Pietrini et al., 2000; Vlassenko et al., 2006; Yehuda et al., 2009). However, one of the main limitations of the bolus method is the lack of temporal information, which is critical for interpreting brain-state changes. Current methods for determining changes in FDG uptake due to a stimulus are confounded by numerous factors: 1) glucose metabolism may not be at equilibrium over the full time course of the experiment (90 minutes or longer) even though a single metabolic rate is derived; 2) sequential measurements often made days or weeks apart introduce uncontrolled variables (e.g. caffeine, sleep status, blood chemistry changes); and 3) mis-registration can occur across scan sessions.

Contrary to traditional FDG-PET imaging, functional magnetic resonance imaging (fMRI) has a good temporal resolution and can be used to dynamically measure multiple responses within a single imaging session (Kwong et al., 1992; Ogawa et al., 1992). Although widely employed for human brain mapping, blood oxygen level dependent (BOLD) fMRI measurements are not quantitative in an absolute sense. The magnitude of the BOLD response reflects a complex interplay between hemodynamic and metabolic responses (Pike, 2012). Measurements of CMR_{glu}, relative to cerebral blood flow (CBF) and cerebral metabolic rate of oxygen (CMRO₂), are important for understanding brain function. Yet, dynamic measurements have been largely ignored in the literature due to the static nature of the FDG technique (Fox et al., 1988, 1986; Raichle and Mintun, 2006).

In this Example, we demonstrate that FDG PET can be used in a more dynamic manner and is capable of repeatedly detecting changes in glucose utilization within a single FDG imaging session, a method we termed “fPET-FDG”. The fundamental principle behind the fPET-FDG method is to control the delivery of FDG to the blood through continuous infusion. By maintaining a constant plasma supply of FDG, dynamic changes in glucose utilization in response to a stimulus or task can be measured with greater sensitivity than with a bolus method. The infusion concept has been pioneered by Carson and others (Carson et al., 1993) as a method to demonstrate changes in receptor occupancy in a single PET scan. Other experiments following the line of this work have applied infusion paradigms that continuously supply radiotracer to take advantage of the increased sensitivity to brain-state changes and the simplifications associated with data acquisition in a single session. Recently, constant infusion of FDG has been applied to dynamically measure the effect of a photodynamic therapy in tumor treatment in mice (Bérard et al., 2006; Cauchon et al., 2012).

The aim of our study reported in this Example is to develop a new method using infusion of FDG to provide better sensitivity to dynamic changes in FDG signal to be used as a human neuroimaging tool. We hypothesized that relative changes in FDG metabolism could be quantified without being confounded by hemodynamic responses, and that the improved temporal resolution would enable multi-task challenges within a single session, thus fundamentally improving PET FDG capabilities. We also hypothesized that the FDG signal using our fPET-FDG protocol could be processed with a modified fMRI pipeline to get statistical maps of the brain activations. In addition to the human neuroimaging studies we report, we also applied a hypercapnic stimulation while acquiring fPET-FDG data concurrently in baboons in order to verify that FDG signal changes are not sensitive to CBF changes.

### METHODS

### Data Acquisition

### PET/MRI Acquisition

All studies involving human subjects were reviewed and approved by the Institutional Review Board (IRB) at Massachusetts General Hospital. All subjects provided written, informed consent in accordance with the Human Research Committee at Massachusetts General Hospital. The imaging studies were performed on a 3-T Tim MAGNETOM Trio MR scanner (Siemens Healthcare, Inc) with an MR-compatible BrainPET insert (Siemens). Three-dimensional (3D) coincidence event data were collected and stored in a list-mode format. Magnetic resonance imaging was performed using two concentric head coils: an outer circularly polarized transmit coil and an inner 8-channel receive-only coil specially designed for the BrainPET with considerations for 511 keV photon attenuation properties.

### PET Imaging Protocol

FDG in saline solution was administered intravenously at a constant infusion rate of 0.01 ml/s for 90 min to healthy subjects (1 female/2 males, mean age 32±2 years) using a Medrad® Spectra Solaris syringe pump (initial activity=5±0.6 mCi). Venous blood samples were collected every 10 minutes from the other arm of all subjects during scanning, then centrifuged to obtain plasma, and aliquoted in a gamma counter that had been calibrated to the PET scanner to measure the venous activity during the experiment.

For reconstruction, the list mode files were sorted into line-of-response space and further compressed into sinogram space. The PET data were reconstructed with a standard 3D ordinary Poisson ordered-subset expectation maximization algorithm using both prompt and variance-reduced random coincidence events as well as normalization, scatter, and attenuation sinograms. The attenuation correction maps were derived from an individual's structural MRI using an MR-based attenuation correction method. The fPET-FDG data were binned into 90 one-minute frames. The data were reconstructed with an isotropic voxel size of 1.25-mm into a volume consisting of 153 transverse slices of 256×256 pixels. All volumes were smoothed using a 3D filter with a 3-mm isotropic Gaussian kernel down-sampled to 76 slices with 128×128 voxels (2.5×2.5×2.5 mm³).

### MRI Imaging Protocol

Anatomical studies consisted of a high-resolution T1-weighting acquisition using multi-echo MPRAGE (TR=2530 ms, TE₁/TE₂/TE₃/TE₄=1.64/3.5/5.36/7.22 ms, TI=1200 ms, flip angle=7°, and 1 mm isotropic) that was used to derive the PET attenuation map. Slice geometries were parallel to the anterior commissure-posterior commissure (AC-PC) plane.

### Visual Stimulation

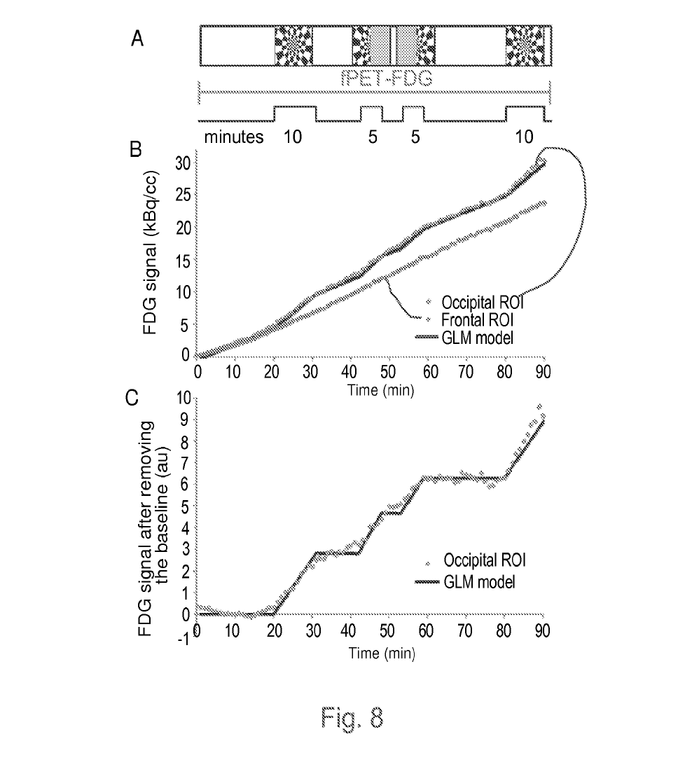

The human visual system, a standard and robust test bed for new neuroimaging techniques, was used to demonstrate that PET FDG studies can operate in a temporally dynamic mode. To induce hemodynamic and metabolic responses, we periodically presented a conventional visual stimulus consisting of an 8-Hz counterphase flickering “checkerboard” pattern (Polimeni et al., 2010) as shown in FIG. 8.A. The stimulus was projected onto a screen mounted at the end of the magnet bore, and subjects viewed the stimulus through a mirror. Three different types of visual stimuli were used: full-field checkerboard, presented twice for a duration of 10 minutes each, and hemi-field checkerboard stimuli (right and left) each presented for a duration of 5 minutes (FIG. 8.A).

Principles of the fPET-FDG Method
Relationship of fPET-FDG to CMR_{glu}

The aim of our experimental design was to dynamically measure changes in FDG metabolism in response to multiple visual stimuli during one FDG infusion. In this section, we outline our mathematical approximation for the resulting FDG signal. Our treatment of data relates the slope of the tissue time-activity curve from FDG infusion to a rate of metabolism and by extension to CMR_{glu}. To give the discussion context, we first point out the strengths and caveats associated with traditional FDG kinetic models.

One classic analysis of FDG kinetics employs an irreversible two-tissue compartment model, which forms the basis of the relationship between the slope of our fPET-FDG method and CMR_{glu}. FDG is trapped in the tissue after initial phosphorylation, such that the reverse rate constant k₄ is small and approximated as null for the duration of experiment (Sokoloff et al., 1977). Using this compartmental model, the following differential equations describe the system (Phelps et al., 1979):

Ċₜ=Ċₘ+Ċ_{f} [1]

Ċₘ=k₃C_{f} [2]

Ċ_{f}=K₁Cₚ−(k₂±k₃)C_{f} [3]

where Cₜ (total tissue concentration) is the sum of Cₘ and C_{f}. Cₘ represents the metabolized (phosphorylated) FDG concentration and C_{f} represents the free/unmetabolized FDG concentration.

In the classic treatment, it is assumed that glucose metabolism measured by FDG is at steady state. The assumption simplifies the kinetic model solution, but comes with the caveat that the assumption is rarely verified or even verifiable using the bolus method. The deviation from steady-state cannot often be extracted from the data to begin to assess what error is introduced through the mathematical simplification. In fact, the bolus method and steady state assumption have been applied in many cases where it is clear that the steady-state assumption is not valid; for instance, acute drug injections produce an evolving physiological response but have been assessed assuming a steady-state (London E D et al., 1990).

In our approach to infusion FDG, we have elected a similar steady-state approximation with the full recognition that the assumption is violated. In fact, any functional imaging technique will violate the steady-state assumption by virtue that changes in metabolism are induced. Given that FDG was infused at a constant rate during the fPET studies herein, we assumed that after an initial equilibration period (discussed later), the freely available FDG for metabolism at rest is nearly constant, hence Ċ_{f}=0, and Ċₜ=k₃C_{f}=Ċₘ. Over the course of the entire imaging session, the plasma concentration does change, but over the shorter times of visual stimulation the plasma concentration is reasonably constant.

When making the steady-state approximation, the equation above can be ‘corrected’ to obtain CMR_{glu} using the lumped constant (Graham et al., 2002; Hasselbalch et al., 2001; Reivich et al., 1985), which converts FDG metabolic rate to glucose and Cₐ⁰, which is a measurement of plasma glucose:

$${CMR}_{glu} = \frac{{C}_{a}^{0}}{( LC ) ( {C}_{p} )} {\dot{C}}_{m} \qquad [ 4 ]$$

Hence, the tissue derivative is proportional to CRM_{glu} at equilibrium, corrected for the lumped constant and the blood concentration according to the steady-state assumption above (Morris et al., 2004). It is worth noting that lumped constant values vary across studies (0.52 to 0.89) and that absolute measurements have additional ambiguities and assumptions (Graham et al., 2002). Since the potential ‘error’ introduced in the choice of lumped constant is so large, many simply use the ‘rate of FDG metabolism’ as a surrogate for CMR_{glu}.

Changes in the slope of the time activity curve of the tissue FDG concentrations are thus proportional to changes in the rate of FDG metabolism and by extension changes in CMR_{glu} using a steady state approximation. During initiation of a task, the compartmental equilibrium concentrations, such as C_{f}, may be disrupted transiently. If we assume that during the task, a new steady state (different from rest) will be reached, then again

$$\frac{d {C}_{f}}{d t} = 0$$

with the transition between states occurring asymptotically. Therefore, we can again approximate CMR_{glu} during the task using the slope of the time activity curve. The % change between slopes measured during two states (rest and task) is thus equal to % change in CMR_{glu}, when Cₐ⁰ and Cₚ are constant.

Since

$$\frac{d {C}_{f}}{d t}$$

cannot be measured, we assessed the steady state assumption through comparisons of our measured data to simulations. Thus, for the infusion method we have the added benefit that we can determine the extent to which the steady state assumption is an approximation. For any functional study based upon FDG uptake, the steady state will be violated, and so results need to be interpreted through compartment models, as in other PET experiments such as displacement studies of receptor-targeted ligand by endogenous neurotransmitter.
CMR_{glu} Maps Derived from the FDG Slope: Subject and Group Level

Aiming to validate our model, we derived CMR_{glu} maps for subjects to compare the values we get with values from the literature. The brain voxel-wise time activity curves for FDG uptake were divided by the interpolated venous blood plasma radioactivity concentrations. Maps of the time activity curve slopes were generated voxel-by-voxel using a linear regression from this blood-normalized FDG signal. CMR_{glu} maps were derived from equation 4 of Example 2 using this slope map with the measured blood glucose concentration (in mmol/L) for each subject and a lumped constant of 0.89 (Graham et al., 2002). An average group FDG slope map was then computed based upon data from the three subjects.

Simulation of the fPET-FDG Signal in Response to a Change in k₃

In order to compare experimental data with theoretical models, simulations were performed using equations [1]-[3] of Example 2. The blood was simulated as a constant input with a bi-exponential wash-out function. A gamma function was used to model a rapid neural response in terms of CMR_{glu}, and then kinetic equations described transient fluctuations of compartmental concentrations, the approach to the new equilibrium state, and the total (free plus bound) tissue concentration. Kinetic rate constants K₁=0.1 mL/min, k₂=0.15 min⁻¹, k₃=0.08 min⁻¹, and k₄=0 min⁻¹ were used in the model (Reivich et al., 1985). FDG signal increases from 10% to 50% were simulated with 10% incremental steps, over a 10-minute period in order to span the range observed in our experimental paradigm.

### Individual Results and Group Analysis

fPET-FDG Processing: Subject Level

We processed each subject's fPET-FDG data using a GLM analysis to verify that our method is sensitive enough to map the visual cortex during a visual activation at the individual subject level.

The PET data were smoothed with a 12 mm Gaussian kernel and analyzed using a general linear model (GLM) consisting of 3 different explanatory variables (EV): (i) 2 blocks of the 10 minutes full-field checkerboard paradigm (EV1), (ii) a 5 minute block of left hemi-field checkerboard (EV2) and (iii) a 5 minute block of right hemi-field checkerboard (EV3).

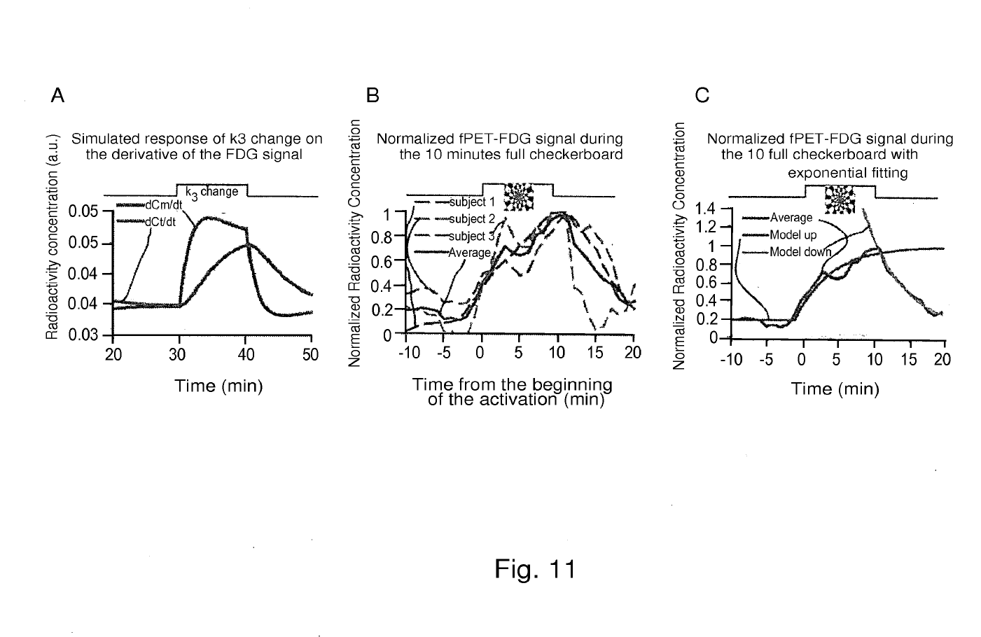

Two separate types of GLM analyses were performed. In one analysis, tissue TACs first were differentiated to form a rate parameter that is closely related to CMR_{glu} (FIG. 11), and then analysis proceeded using a binary (“on-off”) stimulus paradigm similar to fMRI. However, this method exhibits the low signal to noise ratio of individual frames, so an alternative analysis used the original TAC of FDG uptake (FIG. 8B). In this analysis, the hypothesized form of changes in CMR_{glu} produced a set of initial GLM regressor, identically to the first analysis method, and then the regressors were integrated to form the basis set for data analysis using original TACs. Hence, a binary stimulus paradigm for the rate of uptake becomes a series of ramp functions in the analysis of the raw FDG TAC (FIG. 8C).

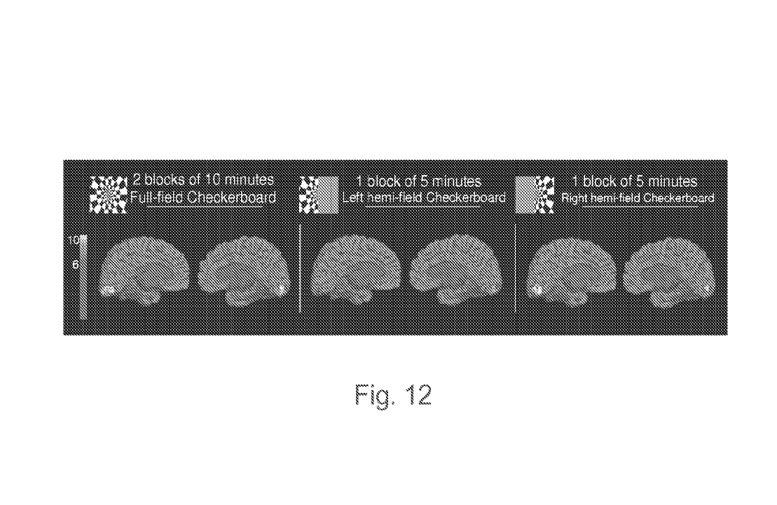

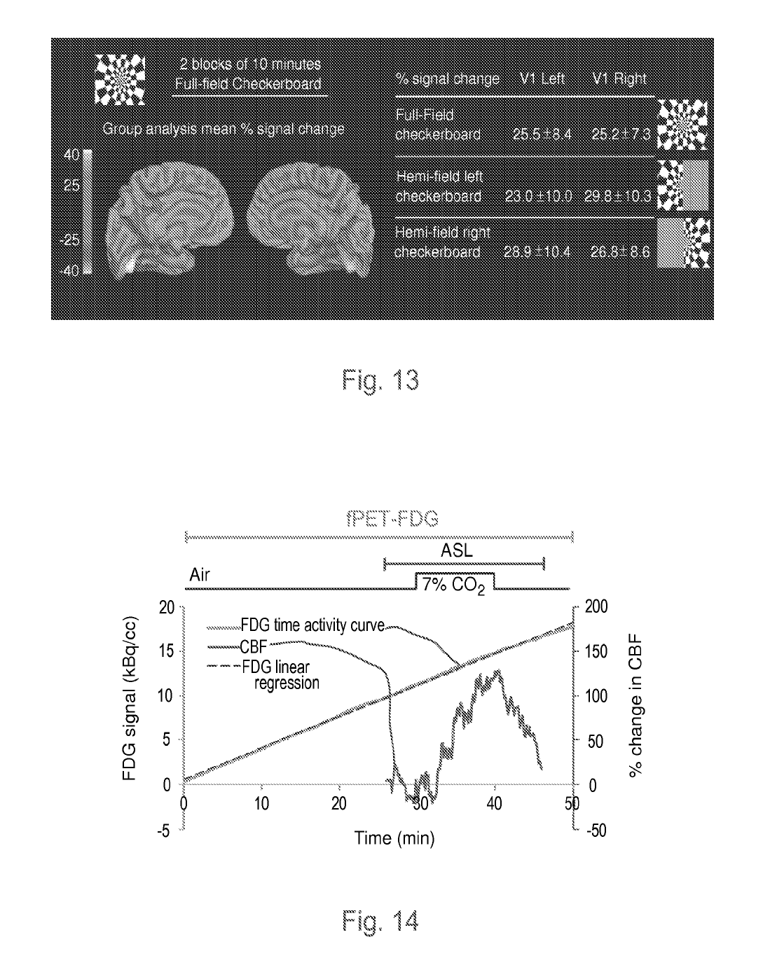

Statistical analyses for FIGS. 12 and 13 used the integral formulation illustrated in FIG. 8, whereas the FDG uptake rate (FIG. 11B) was derived by differentiating the FDG TAC with respect to time.

fPET-FDG Processing: Group Analysis

A second level group analysis was used to validate the statistical significance of our results and also to measure the average changes in FDG metabolism during the visual tasks. The individual subjects' high-resolution anatomical MRI data (acquired concurrently with PET) were coregistered to the MNI152 atlas brain using an affine linear transformation (12 degrees of freedom). The derived transformations were then applied to the dynamic PET data. Statistical significance and effect sizes were determined using a fixed-effects, single-group model. The statistical maps were then projected on surface of the using the FreeSurfer software (Fischl, 2012). Mean percent change±standard deviation for the left and right V1 a priori ROIs (defined anatomically using the Jülich histological atlas) were extracted from FSL for each of the 3 contrasts.

Non-Human Primate (NHP) Experiment with Hypercapnia

To assess the influence of cerebral blood flow changes on FDG signal we administered inhaled carbon dioxide, a strong vasodilatory stimulus, to two baboons (2 females, ˜10-12 kg) simultaneously with fPET-FDG and arterial spin labeling (ASL) MRI for CBF measurements. The protocol was approved by the Institutional Animal Care and Use Committee. Animals were anesthetized with isoflurane (1%) and mechanically ventilated. Physiological parameters were continuously monitored and maintained within the normal range. All images were acquired on the same 3T Siemens TIM-Trio with a BrainPET insert as the human data, using a custom PET-compatible 8-channel array coil. Similar to the human protocol, ˜5 mCi of FDG was continuously infused intravenously at a rate of 0.01 mL/sec for each study. PET data were stored in list mode and binned into 1-minute frames. Dual echo pseudo-continuous arterial spin labeling (pCASL) data were acquired simultaneously (TR/TE1/TE2=4000/12/30 ms, 2.2×2.2×4 mm) (Wey et al., 2010). During a 50 minute dynamic fPET/fMRI scan, a hypercapnic challenge (7% CO₂) was given for 10 minutes between 30 and 40 minutes. All data were motion and slice-time corrected, skull stripped, spatially smoothed and registered to a standard NHP atlas. Percent changes in gray matter CBF were calculated.

### Results

Principles of the fPET-FDG Method

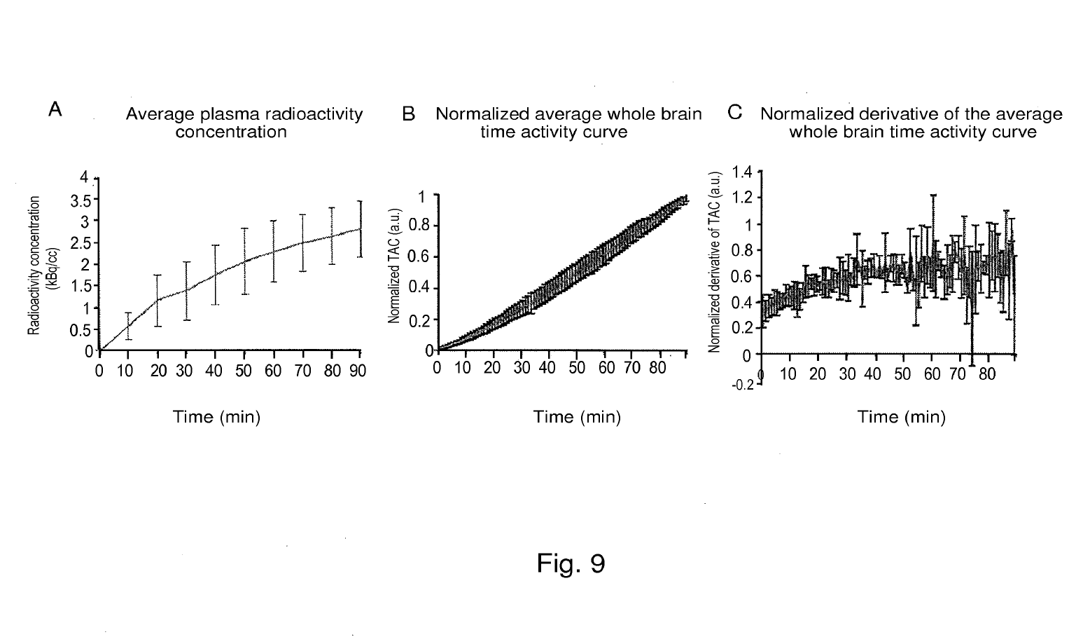

The use of sequential but varied visual stimuli allowed us to confirm that the observed metabolic signal changes were specific to each visual stimulus, and that multiple stimuli (four in this case) can be measured dynamically during a single scan. PET TACs from a single subject are shown in FIG. 8B. Using the activated voxels to define a post-hoc volume-of-interest, the subject's TAC for visual stimulation was created and compared to the frontal cortex, a volume-of-interest that did not significantly respond to the visual stimulus. Changes in FDG signal are easily observed as changes in the TAC slope (FDG utilization rate) during visual stimulus presentation in the activated voxels (occipital region) but not in the frontal cortex (FIG. 8B). These same data are presented after removing the baseline in FIG. 8C together with the GLM model used in the analysis. The baseline term was removed by mathematically modeling a baseline term in the GLM analysis. This baseline term accounts for basal metabolism throughout the experiment, and also for small changes in plasma FDG concentration over the long time course of the overall scan. It is important to note for clarity that we are not using a reference region in this analysis (see Discussion) and that each voxel was processed independently. The TAC for the whole brain (FIG. 9B) demonstrates that the uptake slope of FDG in the brain is quite constant during the entire exam. The derivative of the time activity curve in FIG. 9C also shows that after a delay of about 30 minutes the slope of the FDG signal is stable.

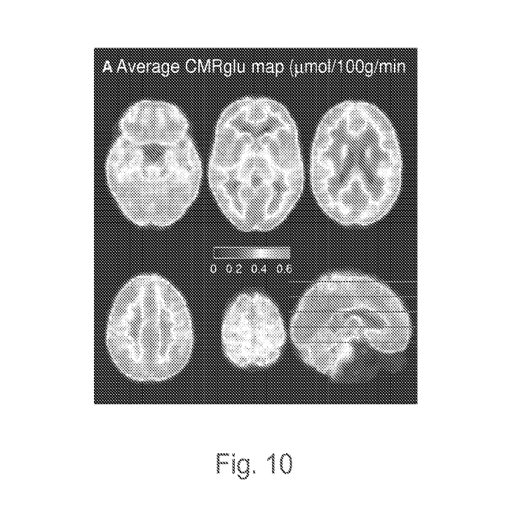

The average concentration of radioactivity in the venous blood from our subjects is shown in FIG. 9. Radioactivity concentration in blood exhibits an initial equilibration followed by a slight increase. Measured blood glucose concentration was 5.6±0.4 mmol/L (mean±sd). Using the FDG signal slope, we calculated average CMR_{glu} values for whole brain from 3 subjects as 0.37±0.02 μmol/100 g/min. Our CMR_{glu} quantification is consistent with published data (Huisman et al., 2012). The average CMR_{glu} map is shown in FIG. 10 at five axial cuts.

Using multi-compartmental simulations (Eqs. 1-3 of Example 2), we modeled the relative dynamic responses of free, bound, and total FDG concentrations. As shown in FIG. 11A, the total tissue concentration, which is the measured experimental quantity, increases more slowly than the bound concentration following a stimulus, due to a corresponding drop in the free concentration. However, the two curves converge toward the end of the stimulus as a new equilibrium is approached. As a way to quantify changes in the metabolized compartment based upon measured changes in total tissue concentration, we compared the area under the curve (AUC) of the tissue and the metabolized TACs following a simulated stimulus. Although the magnitude of the tissue and metabolized compartments do not quite converge in the simulation, the integral of the two curves are much more similar. Across a range of changes in FDG signal from 10%-50%, the AUC ratio was about unity and did not vary significantly (with an averaged AUC ratio of 1.02±0.02 from 5 levels of signal changes), demonstrating that the integral of tissue and metabolized compartmental concentrations are comparable following stimulation.

FIG. 11B shows experimental data for the derivative of the FDG TAC averaged across three subjects. In accordance with the simulation of FIG. 11A, the rate of total tissue uptake increases slowly after stimulation onset and resolves slowly after stimulation offset. Hence, the fPET-FDG signal exhibits the expected dynamics due to visual stimulation, based upon simulated tissue radioactivity concentrations after a brief change in k₃. This Figure also shows that equilibrium is almost completely reached during a brief 10 minute stimulus period (FIG. 11C) and that as predicted from the model, the new equilibrium is approached asymptotically. We are able to estimate the asymptotic value by fitting the data to an exponential response shape, which projects that the magnitude at the end of stimulation is about 98% of the maximum steady-state magnitude. The exponential fit also shows us that the tau of the exponential increase after the beginning of the activation is 4.9 minutes (FIG. 11C) and the tau of the exponential decrease after the activation is 5.6 minutes (FIG. 11C). Hence, an approximately 15 minute spacing between the end of a task and the beginning of another may be necessary to ensure ‘return’ to a baseline equilibrium state before a new activation. The extent to which the exponential increase and decrease during and after activation are governed by physiological changes (versus experimental design) is an area we are actively exploring.

### Individual Results and Group Analysis

We used the GLM analysis to create fPET-FDG activations for the three contrasts of interest (full field checkerboard and right and left hemi-field checkerboard), a representative subject is shown in FIG. 12. Maps show T values, derived from the general linear model, as a measure of the contrast to noise ratio (CNR). Note that CNR values greater than 10 were obtained for each contrast from a single subject in a single session. Confirmation of functional specificity is inherent in our analyses as both hemispheres of the visual system respond equally to the full-field checkerboard (FIG. 12A) whereas the contralateral hemisphere has a higher response to a hemi-field checkerboard (FIG. 12B,C).

Group analysis of the fPET-FDG dataset from three subjects was accomplished using a fixed-effect analysis to determine the average percent change in glucose utilization during each visual stimulus (i.e. left-, right- and full-checkboard) compared to baseline. The percent change map for the full-field checkerboard stimulus at the group level is shown in FIG. 13. The mean percent increase in glucose utilization derived from fPET-FDG for our three subjects was 25% for the full-field checkerboard, 26% for the left hemi-field checkerboard and 28% for the right hemi-field checkerboard (FIG. 10). The absolute changes in FDG utilization as measured by fPET-FDG are consistent with previous studies measuring single response in a two-scan paradigm (Newberg et al., 2005).

Non-Human Primate Experiment with Hypercapnia

In order to assess the impact of CBF changes on fPET-FDG signal, and thus CMR_{glu} estimation, we experimentally modulated CBF in NHP brains using mild hypercapnia. MRI measurements of CBF were obtained simultaneously with fPET-FDG signal. FIG. 14 shows the PET TAC and the ASL CBF data from the gray matter for one baboon. CBF data demonstrated robust signal changes in responses to hypercapnia with a 70% increase on average during the 10 minutes of hypercapnia. Conversely, hypercapnia did not alter FDG uptake rate, indicating that flow effects do not compromise the FDG infusion protocol. This result provides strong empirical evidence that changes in fPET-FDG signals we observe during visual stimuli are not due to changes in blood flow. It is important that we note for both the NHP data and the human data that the tissue response,

$$\frac{d {C}_{t}}{d t} ,$$

(i.e. whole-brain TACs and stimulus-non-responding regions like the FC) are much more constant than the plasma radioactivity (which is changing) would predict.

Fourteen additional participants were scanned (8 male participants and 7 female participants). Of these participants, one participant (female) also received a second scan. All participants received both an MRI and a PET scan simultaneously with motor and/or visual stimulation during the scan. Preliminary results show a colocalization of the motor and/or visual activations in the brain using MRI and PET imaging.

### DISCUSSION

fPET-FDG provides a simple method to observe brain glucose utilization changes dynamically in a single imaging session. We demonstrated that our method enables measurements of changes in FDG signal during multiple tasks within a single imaging session in individual human subjects, a fundamental improvement over the standard bolus technique. Moreover, the hypercapnia data demonstrate that this method exhibits negligible contamination from flow, as expected based upon the low FDG extraction fraction. The processing stream is comparable to other fMRI and PET time-series analyses, thus allowing neuroscientists to adopt this technique easily.

As with any new method, kinetic models and mathematical treatments can be refined. While the steady-state approximation we have applied here is reasonable and supported by our data, we fully acknowledge it is an approximation with certainly limitations. Relaxing the steady-state assumption is a necessary concession to biology in order to accomplish functional brain imaging, and thus analysis models not based upon steady-state approximations are required in order to interpret the data. Further experimental explorations and better models will be beneficial in interpreting the temporal response of FDG using fPET, in the same way models have been created for receptor displacement studies. With our current approximations, there are clear strengths in improved temporal resolution and the ability to perform within-session differential comparisons of FDG metabolism that are imparted by the fPET FDG method.

As a feasibility and proof-of-concept study for fPET-FDG, our results indicate that this method is reliable even at the subject level (FIG. 12). Undoubtedly, additional refinements can be obtained in data acquisition and analysis. Note that we chose a relatively sparse stimulus paradigm (FIG. 8) in order to monitor baseline variations in signal during the infusion protocol. One goal of future studies will be to optimize paradigm designs after characterizing signal and noise frequency components. In order to increase the temporal resolution, the signal to noise ratio (SNR) of the fPET-FDG method could be increased. This optimization might be achieved by modifying the infusion protocol, for example by increasing the infusion rate (radioactivity per time). Additionally, bolus plus continuous infusion paradigms can reach a nearly constant plasma concentration more quickly than infusion without a prior bolus, so that paradigm might offer some additional stability in plasma levels particularly at earlier time points

In data analysis, improvements in motion correction and denoising of the PET data may improve statistical power. Additionally, the signal to noise ratio changes throughout the study; initial analyses using weighted least squares based upon a standard PET noise model (Logan et al., 2001) did not substantially alter results, but further investigations into optimal analysis strategies would be beneficial.

We demonstrated that the basal CMR_{glu} values derived from the slope of FDG signal normalized to blood radioactivity concentration are in very good agreement with values from the literature (average CMR_{glu} values for the all brain of 0.37±0.02 μmol/100 g/min). Hence, we used the slope of the tissue concentration as a function of time as a surrogate for relative CMR_{glu} changes. We are aware that this is an approximation because the transition between steady states and activation period is a function of tracer kinetics. Our simulation results (FIG. 11) show that the metabolized compartment changes more abruptly than the total signal we are acquiring, and our data are in good agreement with this kinetic model.

We have considered many sources of influence and error. One potential source of error could be CBF-induced changes of FDG signals. To address this concern, we demonstrated experimentally that fPET is not influenced measurably by CBF changes. A relatively large increase in CBF (120% change at maximum) due to a hypercapnia stimulus did not induce any measurable change in the FDG signal slope. As such, fPET with FDG appears to be a flow-independent process under normal physiological conditions; however, caution should be exercised under extreme conditions such as hypoglycemia (Schuier et al., 1990), where it is known that the bolus FDG method becomes flow sensitive. From a mathematical point of view, it should be noted that the rate constant for deoxyglucose transport from blood to brain (K₁˜0.1 mL/min) is smaller than the rate constant for its transport back from brain to blood (k₂˜0.15 min⁻¹), and both K₁ and k₂ are greater than k₃ (k₃˜0.08 min⁻¹), the rate constant for its phosphorylation in the brain tissue. These relationships indicate that the blood-brain exchange of deoxyglucose is fast compared to the metabolic rate and so utilization is not limited by supply to the brain through the circulation. Similarly, small changes in CBV also occur during visual activation (Belliveau et al., 1991) and will contribute to the fPET-FDG signal minimally given that each image voxel consists of a small blood component (on average <5%). By measuring blood radioactivity during the infusion protocol (maximum 4 kBq/cc of venous plasma blood), we determined that a 20% change in CBV, would increase the fPET-FDG signal only by 1%. Such a change would make a negligible contribution to our current fPET signal, and could be corrected based upon measured changes in CBF or CBV in situations where greater effects might be considered.

The fPET-FDG method is an operationally straightforward “repurposing” of a fundamental PET index of metabolism using concepts and tools from fMRI. This method can easily be performed on any commercial PET scanner using a widely available and inexpensive radiotracer. Looking forward, the complementary nature of fPET-FDG to fMRI capitalizes on the emerging technology of hybrid MR-PET scanners. In particular, fPET-FDG and emerging quantitative fMRI methods (Buxton, 2012; Hoge, 2012; Pike, 2012) will allow us to simultaneously measure dynamic changes in glucose utilization, hemodynamics and oxygen consumption, addressing vital questions about neuronal and neurovascular relationships across tasks and disease states.

More broadly, our data point towards the capacity of fPET to dynamically image molecular events with the exquisite sensitivity of PET during multi-task challenges. The molecular specificity of a wide range of metabolic and neuroreceptor targeted PET tracers undoubtedly expand fPET beyond measurements of glucose utilization dynamics, for example by improving the temporal resolution of analogous MR-PET dynamic neuroreceptor studies (Mandeville et al., 2013; Sander et al., 2013). We anticipate that fPET will become a modular imaging technique that is extensible to both existing radiotracers and those to be designed specifically for fPET, and one which will provide new temporal information on multifaceted neuronal molecular events, which heretofore have been measured as static ‘state’ functions or an accumulated value. While the temporal resolution of such methods will depend on a number of factors, both technical and biological, dynamic imaging of metabolic or neuroreceptor status will provide an important new dimension to quantify brain functional relationships.

### Bibliography for Example 2

Belliveau, J. W., Kennedy, D. N., Jr, McKinstry, R. C., Buchbinder, B. R., Weisskoff, R. M., Cohen, M. S., Vevea, J. M., Brady, T. J., Rosen, B. R., 1991. Functional mapping of the human visual cortex by magnetic resonance imaging. Science 254, 716-719.
Bérard, V., Rousseau, J. A., Cadorette, J., Hubert, L., Bentourkia, M., Lier, J. E. van, Lecomte, R., 2006. Dynamic Imaging of Transient Metabolic Processes by Small-Animal PET for the Evaluation of Photosensitizers in Photodynamic Therapy of Cancer. J. Nucl. Med. 47, 1119-1126.
Buxton, R. B., 2012. Dynamic models of BOLD contrast. NeuroImage 62, 953-961. doi:10.1016/j.neuroimage.2012.01.012
Carson, R. E., Channing, M. A., Blasberg, R. G., Dunn, B. B., Cohen, R. M., Rice, K. C., Herscovitch, P., 1993. Comparison of bolus and infusion methods for receptor quantitation: application to [¹⁸F]cyclofoxy and positron emission tomography. J. Cereb. Blood Flow Metab. Off. J. Int. Soc. Cereb. Blood Flow Metab. 13, 24-42. doi:10.1038/jcbfm.1993.6
Cauchon, N., Turcotte, E., Lecomte, R., Hasséssian, H. M., Lier, J. E. van, 2012. Predicting efficacy of photodynamic therapy by real-time FDG-PET in a mouse tumour model. Photochem. Photobiol. Sci. 11, 364-370. doi:10.1039/C1PP05294B
Fischl, B., 2012. FreeSurfer. NeuroImage 62, 774-781. doi:10.1016/j.neuroimage.2012.01.021
Fox, P. T., Mintun, M. A., Raichle, M. E., Miezin, F. M., Allman, J. M., Van Essen, D. C., 1986. Mapping human visual cortex with positron emission tomography. Nature 323, 806-809. doi:10.1038/323806a0
Fox, P. T., Raichle, M. E., Mintun, M. A., Dence, C., 1988. Nonoxidative glucose consumption during focal physiologic neural activity. Science 241, 462-464.
Gould, R. W., Gage, H. D., Nader, M. A., 2012. Effects of chronic cocaine self-administration on cognition and cerebral glucose utilization in Rhesus monkeys. Biol. Psychiatry 72, 856-863. doi:10.1016/j.biopsych.2012.05.001
Graham, M. M., Muzi, M., Spence, A. M., O'Sullivan, F., Lewellen, T. K., Link, J. M., Krohn, K. A., 2002. The FDG lumped constant in normal human brain. J. Nucl. Med. Off. Publ. Soc. Nucl. Med. 43, 1157-1166.
Hasselbalch, S. G., Holm, S., Pedersen, H. S., Svarer, C., Knudsen, G. M., Madsen, P. L., Paulson, O. B., 2001. The 18F-fluorodeoxyglucose Lumped Constant Determined in Human Brain From Extraction Fractions of ¹⁸F-fluorodeoxyglucose and Glucose. J. Cereb. Blood Flow Metab. 21, 995-1002. doi:10.1097/00004647-200108000-00012
Hoge, R. D., 2012. Calibrated fMRI. NeuroImage 62, 930-937. doi:10.1016/j.neuroimage.2012.02.022
Huisman, M. C., van Golen, L. W., Hoetjes, N. J., Greuter, H. N., Schober, P., Ijzerman, R. G., Diamant, M., Lammertsma, A. A., 2012. Cerebral blood flow and glucose metabolism in healthy volunteers measured using a high-resolution PET scanner. EJNMMI Res. 2, 63. doi:10.1186/2191-219X-2-63
Kushner, M. J., Rosenquist, A., Alavi, A., Rosen, M., Dann, R., Fazekas, F., Bosley, T., Greenberg, J., Reivich, M., 1988. Cerebral metabolism and patterned visual stimulation: a positron emission tomographic study of the human visual cortex. Neurology 38, 89-95.
Kwong, K. K., Belliveau, J. W., Chesler, D. A., Goldberg, I. E., Weisskoff, R. M., Poncelet, B. P., Kennedy, D. N., Hoppel, B. E., Cohen, M. S., Turner, R., 1992. Dynamic magnetic resonance imaging of human brain activity during primary sensory stimulation. Proc. Natl. Acad. Sci. 89, 5675.
Logan, J., Fowler, J. S., Volkow, N. D., Ding, Y. S., Wang, G. J., Alexoff, D. L., 2001. A strategy for removing the bias in the graphical analysis method. J. Cereb. Blood Flow Metab. Off. J. Int. Soc. Cereb. Blood Flow Metab. 21, 307-320. doi:10.1097/00004647-200103000-00014
London E D, Cascella N G, Wong D F, et al, 1990. Cocaine-induced redoppuction of glucose utilization in human brain: A study using positron emission tomography and [fluorine 18]-fluorodeoxyglucose. Arch. Gen. Psychiatry 47, 567-574. doi:10.1001/archpsyc.1990.01810180067010
Mandeville, J. B., Sander, C. Y. M., Jenkins, B. G., Hooker, J. M., Catana, C., Vanduffel, W., Alpert, N. M., Rosen, B. R., Normandin, M. D., 2013. A receptor-based model for dopamine-induced fMRI signal. NeuroImage 75, 46-57. doi:10.1016/j.neuroimage.2013.02.036
Molina, V., Solera, S., Sanz, J., Sarramea, F., Luque, R., Rodriguez, R., Jiménez-Arriero, M. A., Palomo, T., 2009. Association between cerebral metabolic and structural abnormalities and cognitive performance in schizophrenia. Psychiatry Res. 173, 88-93. doi:10.1016/j.pscychresns.2008.09.009
Morris, E. D., Endres, C. J., Schmidt, K. C., Christian, B. T., Muzic Jr., R. F., Fisher, R. E., 2004. Chapter 23—Kinetic Modeling in Positron Emission Tomography, in: Wernick, M. N., Aarsvold, J. N. (Eds.), Emission Tomography. Academic Press, San Diego, pp. 499-540.
Newberg, A. B., Wang, J., Rao, H., Swanson, R. L., Wintering, N., Karp, J. S., Alavi, A., Greenberg, J. H., Detre, J. A., 2005. Concurrent CBF and CMRGIc changes during human brain activation by combined fMRI-PET scanning. NeuroImage 28, 500-506. doi:10.1016/j.neuroimage.2005.06.040
Ogawa, S., Tank, D. W., Menon, R., Ellermann, J. M., Kim, S. G., Merkle, H., Ugurbil, K., 1992. Intrinsic signal changes accompanying sensory stimulation: functional brain mapping with magnetic resonance imaging. Proc. Natl. Acad. Sci. U.S.A. 89, 5951-5955.
Phelps, M. E., Huang, S. C., Hoffman, E. J., Selin, C., Sokoloff, L., Kuhl, D. E., 1979. Tomographic measurement of local cerebral glucose metabolic rate in humans with (F-18)2-fluoro-2-deoxy-D-glucose: validation of method. Ann. Neurol. 6, 371-388. doi:10.1002/ana.410060502
Pietrini, P., Alexander, G. E., Furey, M. L., Dani, A., Mentis, M. J., Horwitz, B., Guazzelli, M., Schapiro, M. B., Rapoport, S. I., 2000. Cerebral Metabolic Response to Passive Audiovisual Stimulation in Patients with Alzheimer's Disease and Healthy Volunteers Assessed by PET. J. Nucl. Med. 41, 575-583.
Pike, G. B., 2012. Quantitative functional MRI: Concepts, issues and future challenges. NeuroImage 62, 1234-1240. doi:10.1016/j.neuroimage.2011.10.046
Polimeni, J. R., Fischl, B., Greve, D. N., Wald, L. L., 2010. Laminar analysis of 7T BOLD using an imposed spatial activation pattern in human V1. NeuroImage 52, 1334-1346. doi:10.1016/j.neuroimage.2010.05.005
Raichle, M. E., Mintun, M. A., 2006. Brain Work and Brain Imaging. Annu. Rev. Neurosci. 29, 449-476. doi:10.1146/annurev.neuro.29.051605.112819 Reivich, M., Alavi, A., Wolf, A., Fowler, J., Russell, J., Arnett, C., MacGregor, R. R., Shiue, C. Y., Atkins, H., Anand, A., Dann, R., Greenberg, J. H., 1985. Glucose Metabolic Rate Kinetic Model Parameter Determination in Humans: The Lumped Constants and Rate Constants for [18F]Fluorodeoxyglucose and [¹¹C]Deoxyglucose. J. Cereb. Blood Flow Metab. 5, 179-192. doi:10.1038/jcbfm.1985.24
Reivich, M., Kuhl, D., Wolf, A., Greenberg, J., Phelps, M., Ido, T., Casella, V., Fowler, J., Hoffman, E., Alavi, A., Som, P., Sokoloff, L., 1979. The [¹⁸F]fluorodeoxyglucose method for the measurement of local cerebral glucose utilization in man. Circ. Res. 44, 127-137.
Sander, C. Y., Hooker, J. M., Catana, C., Normandin, M. D., Alpert, N. M., Knudsen, G. M., Vanduffel, W., Rosen, B. R., Mandeville, J. B., 2013. Neurovascular coupling to D2/D3 dopamine receptor occupancy using simultaneous PET/functional MRI. Proc. Natl. Acad. Sci. doi:10.1073/pnas.1220512110
Schuier, F., Orzi, F., Suda, S., Lucignani, G., Kennedy, C., Sokoloff, L., 1990.
Influence of plasma glucose concentration on lumped constant of the deoxyglucose method: effects of hyperglycemia in the rat. J. Cereb. Blood Flow Metab. Off. J. Int. Soc. Cereb. Blood Flow Metab. 10, 765-773. doi:10.1038/jcbfm.1990.134
Sokoloff, L., Reivich, M., Kennedy, C., Des Rosiers, M. H., Patlak, C. S., Pettigrew, K. D., Sakurada, O., Shinohara, M., 1977. The [¹⁴C]deoxyglucose method for the measurement of local cerebral glucose utilization: theory, procedure, and normal values in the conscious and anesthetized albino rat. J. Neurochem. 28, 897-916.
Vlassenko, A. G., Rundle, M. M., Mintun, M. A., 2006. Human brain glucose metabolism may evolve during activation: Findings from a modified FDG PET paradigm. NeuroImage 33, 1036-1041. doi:10.1016/j.neuroimage.2006.06.065
Wey, H. Y., Wang, D. J., Duong, T. Q., 2010. Baseline CBF, and BOLD, CBF, and CMRO2 fMRI of visual and vibrotactile stimulations in baboons. J. Cereb. Blood Flow Metab. 31, 715-724.
Yehuda, R., Harvey, P. D., Golier, J. A., Newmark, R. E., Bowie, C. R., Wohltmann, J. J., Grossman, R. A., Schmeidler, J., Hazlett, E. A., Buchsbaum, M. S., 2009. Changes in relative glucose metabolic rate following cortisol administration in aging veterans with posttraumatic stress disorder: an FDG-PET neuroimaging study. J. Neuropsychiatry Clin. Neurosci. 21, 132-143. doi:10.1176/appi.neuropsych.21.2.132

The citation of any document is not to be construed as an admission that it is prior art with respect to the present invention.

Thus, the fPET method described herein is a new technique that provides a sub-minute dynamic monitoring of brain metabolic changes during brain activity in response to physiological changes. While the conventional method for monitoring brain activity is BOLD-fMRI, fPET-FDG has the advantage to be inherently quantitative and mono-parametric, compared to fMRI. fPET-FDG should prove invaluable for testing neurovascular coupling assumptions underlying BOLD fMRI. Moreover, it will augment the utility of PET-FDG as a neuroscience tool.

Our fPET-FDG method should have broad applicability in cognitive neuroscience and beyond (i.e., the method is not limited to the brain). As a functional imaging technique, it can be used to obtain voxel-wise activation and/or deactivation maps during sensory and cognitive paradigms (visual, auditory, working memory), and also physiological paradigms (hypercapnia, hypoxia). Moreover, it could also be used to test drugs effects on brain metabolism dynamically in a single scan session. In addition, fPET-FDG method can be used to determine proper tumor margin resection locations. Furthermore, this technique could be employed in clinical research protocols to assess brain metabolism deficit in neurodegenerative patients during working memory challenges.

Our method only requires positron emitter (e.g., FDG) infusion and a PET scan. It does not require MRI data, which could be of great interest for patients who cannot undergo MRI (metals implants, monitoring, claustrophobia) or for research experiments being transferable into an MRI with difficulty (transcranial magnetic stimulation, electrodes). The methods described herein can be accomplished with a PET-only or PET-CT scanner. This technique is easily transferable to the clinical settings once developed for specific diagnostic, staging or prognostic applications.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the embodiments contained herein.

What is claimed is:

1. An emission tomography system for acquiring a series of medical images of a subject during an imaging process using a radiotracer, the system comprising:
a plurality of detectors configured to be arranged about the subject to acquire gamma rays emitted from the subject over a time period relative to an administration of the radiotracer to the subject and communicate signals corresponding to acquired gamma rays;
a stimulus system configured to track a stimulus associated with the subject over the time period and communicate a stimulus feedback;
a data processing system configured to receive the signals from the plurality of detectors and the stimulus feedback from the stimulus system and correlate the signals with information about the stimulus associated with the subject; and
a reconstruction system configured to receive correlated signals and information about the stimulus from the data processing system and reconstruct therefrom a series of medical images of the subject showing a physiological response of the subject to the stimulus over the time period.

2. The system of claim 1 wherein the stimulus includes at least one of a visual stimulus, an auditory stimulus, and a mechanical stimulus.

3. The system of claim 1 wherein the stimulus includes at least two of a visual stimulus, an auditory stimulus, and a mechanical stimulus.

4. The system of claim 1 wherein the stimulus includes a visual stimulus.

5. The system of claim 1 wherein:
the stimulus is a sensory stimulus directed to the brain of the subject, and
the detectors are arranged to acquire gamma rays emitted from the brain of the subject, and
the reconstruction system receives correlated signals and information about the stimulus from the data processing system and reconstructs therefrom a series of medical images of the brain of the subject.

6. The system of claim 1, wherein the stimulus is a sensory stimulus initially targeted to one or more senses of the subject.

7. The system of claim 6, wherein the one or more senses of the subject are selected from the group consisting of sight, hearing, taste, smell, touch, and proprioception.

8. The system of claim 6, wherein one of the one or more senses is sight.

9. The system of claim 1 wherein the radiotracer comprises a positron emitter.

10. The system of claim 1 wherein the radiotracer comprises at least one of ¹¹C, ¹³N, ¹⁵O, ¹⁸F, ⁶⁴Cu, ⁶⁸Ga, ⁷⁵Br, ⁷⁸Br, ⁸²Rb, ⁸⁹Zr, ¹²¹I, and ¹²⁴I.

11. The system of claim 1 wherein the radiotracer includes ¹⁸F-fluorodeoxyglucose (FDG).

12. The system of claim 1 wherein the physiological response of the subject to the stimulus includes activation of portions of a brain of the subject in response to the stimulus.

13. The system of claim 1 further comprising an administration system configured to deliver the radiotracer to the subject at a controlled rate.

14. The system of claim 1 further comprising an administration system configured to deliver the radiotracer to the subject at a constant rate.

15. The system of claim 1 further comprising a magnetic resonance imaging device for acquiring a second series of medical images of the subject.

16. The system of claim 15 wherein the series of medical images of the subject and the second series of medical images of the subject are acquired concurrently.

17. The system of claim 1 wherein:
the reconstruction system is configured to derive a parametric map from time series data.

18. The system of claim 1 wherein:
the reconstruction system is configured to derive cerebral metabolic rate of glucose (CMR_{glu}) from time series data.

19. A method of dynamically imaging a subject over a set time period by emission tomography, the method comprising:
(a) administering a radiotracer to the subject continuously over the set time period;
(b) directing one or more tracked stimuli to the subject during the set time period;
(c) using a plurality of detectors to detect gamma rays emitted from the subject and to communicate signals corresponding to the detected gamma rays over the set time period;
(d) correlating the signals with information about the tracked stimuli directed to the subject; and
(e) reconstructing from this correlation a series of medical images of a region of interest of the subject.

20. The method of claim 19 wherein step (b) comprises directing one or more tracked sensory stimuli to the brain of the subject.

21. The method of claim 19 wherein the physiological response of the subject to the stimulus includes activation of portions of a brain of the subject in response to the stimulus.

22. The method of claim 19, wherein the series of medical images show a physiological response to the tracked stimuli over the time period.

23. The method of claim 19 wherein the tracked stimuli include at least one of a visual stimulus, an auditory stimulus, and a mechanical stimulus.

24. The method of claim 19 wherein the tracked stimuli include at least two of a visual stimulus, an auditory stimulus, and a mechanical stimulus.

25. The method of claim 19 wherein the tracked stimuli include a visual stimulus.

26. The method of claim 19 wherein the tracked stimuli are directed to the brain of the subject and are initially targeted to one or more of the senses of the subject.

27. The method of claim 26, wherein the one or more senses of the subject are selected from the group consisting of sight, hearing, taste, smell, touch, and proprioception.

28. The method of claim 26, wherein one of the one or more senses is sight.

29. The method of claim 19 wherein the radiotracer comprises a positron emitter.

30. The method of claim 19 wherein the radiotracer comprises at least one of ¹¹C, ¹³N, ¹⁵O, ¹⁸F, ⁶⁴Cu, ⁶⁸Ga, ⁷⁵Br, ⁷⁸Br, ⁸²Rb, ⁸⁹Zr, ¹²¹I, and ¹²⁴I.

31. The method of claim 19 wherein the radiotracer includes ¹⁸F-fluorodeoxyglucose (FDG).

32. The method of claim 19 wherein the radiotracer is administered to the subject at a controlled rate during the time period.

33. The method of claim 19 wherein the radiotracer is administered to the subject at a constant rate during the time period.

34. The method of claim 19 further comprising:
acquiring a second series of medical images using magnetic resonance imaging.

35. The method of claim 19 further comprising:
concurrently acquiring a second series of medical images using magnetic resonance imaging.

36. The method of claim 19 further comprising:
deriving a parametric map from time series data.

37. The method of claim 19 further comprising:
deriving cerebral metabolic rate of glucose (CMR_{glu}) from time series data.
